(19) United States
(12) Patent Application Publication
(10) Pub. No.: US 2018/0187218 A1
(43) Pub. Date: Jul. 5, 2018

(54) PROCESSING BIOMASS

(71) Applicant: Xyleco, Inc., Wakefield, MA (US)

(72) Inventors: Marshall Medoff, Wakefield, MA (US); Thomas Craig Masterman, Rockport, MA (US); Christopher G.F. Cooper, Rehoboth, MA (US)

(21) Appl. No.: 15/886,123

(22) Filed: Feb. 1, 2018

Related U.S. Application Data
(63) Continuation of application No. 15/434,779, filed on Feb. 16, 2017.
Application No. 15/434,779 is a division of application No. 14/439,799, filed on Apr. 30, 2015.
Application No. 14/439,799 is a continuation-in-part of application No. PCT/US2014/035467, filed on Apr. 25, 2014.
Application No. PCT/US2014/035467 is a continuation-in-part of application No. PCT/US2014/035469, filed on Apr. 25, 2014.
(60) Provisional application No. 61/941,771, filed on Feb. 19, 2014.
(60) Provisional application No. 62/014,718, filed on Jun. 20, 2014.

(51) Int. Cl. C12P 7/10; C12P 19/02; C12P 19/14

(52) U.S. Cl. CPC C12P 7/10; C12P 2201/00; C12P 19/14; C12P 19/02

(57) ABSTRACT

Biomass feedstocks (e.g., plant biomass, animal biomass, and municipal waste biomass) are processed to produce useful products, such as fuels. For example, systems are described that can be useful in enhancing sugar yields from biomass.

### RELATED APPLICATIONS

This application is a continuation application of U.S. application Ser. No. 15/434,779, filed Feb. 16, 2017, which is a divisional application of U.S. application Ser. No. 14/439,799, filed Apr. 30, 2015, which is a U.S. National Phase of PCT Application Serial No. PCT/US14/59970, filed Oct. 9, 2014, which claims benefit of U.S. Provisional Application Ser. No. 61/941,771, filed Feb. 19, 2014, and is a continuation-in-part application of PCT/US14/035467, filed Apr. 25, 2014 and PCT/US14/035469, filed Apr. 25, 2014, and claims benefit of U.S. Provisional Application Ser. No. 62/014,718, filed Jun. 20, 2014.

### BACKGROUND OF THE INVENTION

Many potential lignocellulosic feedstocks are available today, including agricultural residues, woody biomass, municipal waste, oilseeds/cakes and seaweed, to name a few. At present, these materials are often under-utilized, being used, for example, as animal feed, biocompost materials, burned in a co-generation facility or even landfilled.

Lignocellulosic biomass includes crystalline cellulose fibrils embedded in a hemicellulose matrix, surrounded by lignin. This produces a compact matrix that is difficult to access by enzymes and other chemical, biochemical and/or biological processes. Cellulosic biomass materials (e.g., biomass material from which the lignin has been removed) are more accessible to enzymes and other conversion processes, but even so, naturally-occurring cellulosic materials often have low yields (relative to theoretical yields) when contacted with hydrolyzing enzymes. Lignocellulosic biomass is even more recalcitrant to enzyme attack. Furthermore, each type of lignocellulosic biomass has its own specific composition of cellulose, hemicellulose and lignin.

### SUMMARY

In general, methods, equipment and systems are disclosed herein for reducing the recalcitrance of biomass materials (e.g., including cellulose, lignocellulose, starches). Methods, equipment and systems for saccharifying biomass materials and further processing the saccharified biomass to other products such as sugars, sugar alcohols, alcohols, enzymes and carboxylic acids are also described. Recalcitrance reduction methods include heating of the material to a high temperature and maintaining the material at the high temperature for a time sufficient to reduce its recalcitrance and thus make the material more susceptible to further processing (e.g., biochemical processes such as saccharification and fermentation). Other recalcitrance methods include irradiation, steam explosion, pyrolysis, oxidation and sonication. These methods can be applied before and/or after heating in any combination and optionally repeatedly.

In one aspect, the invention relates to methods, equipment and systems for processing a biomass including heating a slurry that comprises a reduced recalcitrance lignocellulosic material to a temperature greater than about 120 DEG C. for a time sufficient to further reduce the recalcitrance of the material. Optionally the slurry comprises a lignocellulosic material that has been irradiated with between about 1 and 100 Mrad of ionizing radiation (e.g., between about 10 and about 50 Mrad, between about 20 and about 40 Mrad). Optionally, the slurry comprises at least about 10 wt % solids (e.g., at least about 20 wt %, at least 30 wt %, at least about 40%, at least about 50 wt %). The reduced recalcitrance material can be heated for a time sufficient to swell the material to at least about 5 vol. % higher than an un-heated lignocellulosic material (e.g., at least about 10 vol. %, at least about 20 vol %, at least about 30 vol. %, at least about 40 vol. %, at least about 50 vol. %). The reduced recalcitrance material can additionally or optionally be heated for a time sufficient to reduce the crystallinity of the material by at least 10% (e.g., at least 20%, at least 30%, at least 40%, at least 50%). In some embodiments the reduced recalcitrance material is initially at a temperature below about 50 DEG C. and reaches a temperature above about 120 DEG C. in less than about 20% of the total time the biomass material is held at the temperature above about 120 DEG C. (e.g., less than about 10%, less than about 5%, less than about 1%). In some embodiments the time to reach the temperature above about 120 DEG C. is less than about 6 min (e.g., less than about 3 min, less than about 1 min, less than about 30 seconds, less than about 10 seconds) and the time the material is held at the temperature above about 120 DEG C. is at least 10 min (e.g., at least about 20 min, at least about 30 min, at least about 1 hour, at least about 4 hours, at least about 8 hours, at least about 12 hours). In some implementations heating the slurry includes heating by steam injection heating (e.g., externally modulated steam injection, internally modulated steam injection). In some other implementations, heating includes heating the slurry in a tube reactor (e.g., configured as a heated screw conveyor). Optionally, heating includes heating the slurry utilizing indirect heating (e.g., utilizing a heated screw conveyor, utilizing a heated pressure cooker/tube reactor). In some implementations, heating includes heating the slurry in a tube reactor while agitating the slurry, for example wherein agitating comprises mixing with a mechanical mixer selected from the group consisting of an auger mixer, a jet mixer, a recirculating pump and combinations thereof. In some implementations the material is cooled in a flash tank after heating the material (e.g., to a temperature between about 90 and 110 DEG C.). For example, the material can be cooled utilizing a cooling fluid fed heat exchanger (e.g., to a temperature between about 20 and about 80 DEG C., between about 30 and about 70 DEG C.). In some implementations, the material is saccharified (e.g., utilizing an enzyme such as a cellulase and/or acid). In some implementations the material is contacted with an enzyme or organism. Optionally, the lignocellulosic material is selected from the group consisting of wood, particle board, forestry wastes (e.g., sawdust, aspen wood, wood chips), grasses, (e.g., switchgrass, miscanthus, cord grass, reed canary grass), grain residues, (e.g., rice hulls, oat hulls, wheat chaff, barley hulls), agricultural waste (e.g., silage, canola straw, wheat straw, barley straw, oat straw, rice straw, jute, hemp, flax, bamboo, sisal, abaca, corn cobs, corn stover, soybean stover, corn fiber, alfalfa, hay, coconut hair), sugar processing residues (e.g., bagasse, beet pulp, agave bagasse), algae, seaweed, manure, sewage, and mixtures of any of these. Optionally, the lignocellulosic material is fermented and then heated a second time to a temperature greater than about 120 DEG C. for a time sufficient to further reduce the recalcitrance of the material. In some implementations, the lignocellulosic material is heated at a rate of between about 340 DEG C.·Kg/min and about 10,000,000 DEG C.·Kg/min (e.g., between about 100,000 and about 500,000 DEG C.·Kg/min). Optionally, the reduced recalcitrance material has an average particle size between about 0.25 mm and about 3 mm (e.g., between about 0.5 mm and about 2 mm) prior to being heated.

Another aspect of the invention relates to systems, equipment and methods for processing a biomass material including heating a reduced recalcitrance lignocellulosic material to a temperature greater than about 120 DEG C. using steam, and forming a slurry by combining the steam and the reduced recalcitrance lignocellulosic material, wherein the steam condenses and wets the reduced recalcitrance lignocellulosic material by condensation onto the reduced recalcitrance lignocellulosic material (e.g., interior or exterior surfaces thereof). Optionally the reduced recalcitrance lignocellulosic material is heated at a rate of between about 340 DEG C.·Kg/min and about 10,000,000 DEG C.·Kg/min (e.g., between about 100,000 and about 500,000 DEG C.·Kg/min). In some implementations, the slurry is held at the temperature greater than about 120 DEG C. for at least 1 min (e.g., at least 5 min, at least 10 min, at least 20 min, at least 30 min, at least 1 hour, at least 4 hours, at least 8 hours, at least 12 hours). Optionally, the reduced recalcitrance lignocellulosic material comprises a slurry of at least about 10 wt % solids (e.g., at least about 20 wt %, at least 30 wt %, at least 40%, at least 50 wt %). Optionally, the methods include cooling the slurry to a temperature between about 20 and about 80 DEG C. (e.g., between about 30 and about 70 DEG C.) In some implementations, the steam and reduced recalcitrance lignocellulosic material are combined utilizing a Jet Cooker. The slurry can be collected in a tube reactor that is agitated internally utilizing a mechanical agitator selected from the group consisting of an auger mixer, a jet mixer, a recirculating pump, and combinations thereof. In some implementations, the methods includes contacting the reduced recalcitrance lignocellulosic material with an organism or enzyme. Optionally, the reduced recalcitrance lignocellulosic material is made by irradiating a cellulosic or lignocellulosic material with between about 10 and 50 Mrad of ionizing radiation (e.g., about 20 and about 40 Mrad).

In yet another aspect, the invention relates to a method for processing biomass that includes producing a sugar solution by enzymatically saccharifying a cellulosic or lignocellulosic material. The recalcitrance of the cellulosic or lignocellulosic material has been reduced by steam heating the material to a target temperature and irradiating the cellulosic or lignocellulosic material. For example, recalcitrance may be reduced by irradiating the material first and then heating it, or heating the material and subsequently irradiating it. Optionally, the cellulosic or lignocellulosic material is held at the target temperature for between about 1 and about 240 min. Optionally the cellulosic or lignocellulosic material has been irradiated with an electron beam and receives a dose of irradiation between about 10 and about 50 Mrad. In some implementations the target temperature is between about 120 and 160 DEG C. Optionally, the material has been irradiated with between about 20 and about 40 Mrad, the final temperature is between about 140 and about 180 DEG C., and the temperature is held at the final temperature for between about 1 min and about 60 min. In some implementations, the sugar solution is fermented.

The heating recalcitrance reduction methods described herein, such as the heating method or irradiation in combination with heating, can be advantageous because they provide an efficient method for recalcitrance reduction and enhancing sugar yields from biomass. In particular, the methods are efficient in heating large quantities of biomass material to high temperatures in a short period of time, enabling implementation of the method at a plant scale. In addition, the methods can be synergistically combined with other recalcitrance reduction methods so that a high yield of products can be derived from a feedstock biomass.

Implementations of the embodiments can optionally include one or more of the following summarized features. In some implementations, the selected features can be applied or utilized in any order while in other implementations a specific selected sequence is applied or utilized. Individual features can be applied or utilized more than once in any sequence and even continuously. In addition, an entire sequence, or a portion of a sequence, of applied or utilized features can be applied or utilized once, repeatedly or continuously in any order. In some optional implementations, the features can be applied or utilized with different, or where applicable, the same set or varied, quantitative or qualitative parameters as determined by a person skilled in the art. For example, parameters of the features such as size, individual dimensions (e.g., length, width, height), location of, degree (e.g., to what extent such as the degree of recalcitrance), duration, frequency of use, density, concentration, intensity and speed can be varied or set, where applicable, as determined by a person of skill in the art.

Features, for example, include: a method for processing a biomass; heating a slurry comprising a reduced recalcitrance lignocellulosic materials to a temperature greater than about 120 DEG C. for a time sufficient to further reduce the recalcitrance of the material; a slurry comprises a lignocellulosic material that has been irradiated with between about 1 and 100 Mrad of ionizing radiation; a slurry comprises a lignocellulosic material that has been irradiated with between about 20 and 40 Mrad of ionizing radiation; a slurry comprises at least about 10 wt. % solids; a slurry comprises at least about 30 wt. % solids; a slurry comprises at least about 40 wt % solids; a slurry comprises at least about 50 wt % solids; a reduced recalcitrance material is heated for a time sufficient to swell the material to at least about 5 vol. % higher than an un-heated lignocellulosic material; a reduced recalcitrance material is heated for a time sufficient to swell the material to at least about 10 vol. % higher than an un-heated lignocellulosic material; a reduced recalcitrance material is heated for a time sufficient to swell the material to at least about 20 vol. % higher than an un-heated lignocellulosic material; a reduced recalcitrance material is heated for a time sufficient to swell the material to at least about 30 vol. % higher than an un-heated lignocellulosic material; a reduced recalcitrance material is heated for a time sufficient to swell the material to at least about 40 vol. % higher than an un-heated lignocellulosic material; a reduced recalcitrance material is heated for a time sufficient to swell the material to at least about 50 vol. % higher than an un-heated lignocellulosic material; a reduced recalcitrance material is heated for a time sufficient to reduce the crystallinity of the material by at least 10%; a reduced recalcitrance material is heated for a time sufficient to reduce the crystallinity of the material by at least 20%; a reduced recalcitrance material is heated for a time sufficient to reduce the crystallinity of the material by at least 30%; a reduced recalcitrance material is heated for a time sufficient to reduce the crystallinity of the material by at least 40%; a reduced recalcitrance material is heated for a time sufficient to reduce the crystallinity of the material by at least 50%; a reduced recalcitrance material is initially at a temperature below about 50 DEG C. and reaches a temperature above about 120 DEG C. in less than about 20% of the total time the biomass material is held at the temperature above about 120 DEG C.; a reduced recalcitrance material is initially at a temperature below about 50 DEG C. and reaches a temperature above about 120 DEG C. in less than about 10% of the total time the biomass material is held at the temperature above about 120 DEG C.; a reduced recalcitrance material is initially at a temperature below about 50 DEG C. and reaches a temperature above about 120 DEG C. in less than about 5% of the total time the biomass material is held at the temperature above about 120 DEG C.; a reduced recalcitrance material is initially at a temperature below about 50 DEG C. and reaches a temperature above about 120 DEG C. in less than about 1% of the total time the biomass material is held at the temperature above about 120 DEG C.; a reduced recalcitrance material is initially at a temperature below about 70 DEG C. and reaches a temperature above about 120 DEG C. in less than about 20% of the total time the biomass material is held at the temperature above about 120 DEG C.; a reduced recalcitrance material is initially at a temperature below about 70 DEG C. and reaches a temperature above about 120 DEG C. in less than about 10% of the total time the biomass material is held at the temperature above about 120 DEG C.; a reduced recalcitrance material is initially at a temperature below about 70 DEG C. and reaches a temperature above about 120 DEG C. in less than about 5% of the total time the biomass material is held at the temperature above about 120 DEG C.; a reduced recalcitrance material is initially at a temperature below about 70 DEG C. and reaches a temperature above about 120 DEG C. in less than about 1% of the total time the biomass material is held at the temperature above about 120 DEG C.; a reduced recalcitrance material is initially at a temperature below about 90 DEG C. and reaches a temperature above about 120 DEG C. in less than about 20% of the total time the biomass material is held at the temperature above about 120 DEG C.; a reduced recalcitrance material is initially at a temperature below about 90 DEG C. and reaches a temperature above about 120 DEG C. in less than about 10% of the total time the biomass material is held at the temperature above about 120 DEG C.; a reduced recalcitrance material is initially at a temperature below about 90 DEG C. and reaches a temperature above about 120 DEG C. in less than about 5% of the total time the biomass material is held at the temperature above about 120 DEG C.; a reduced recalcitrance material is initially at a temperature below about 90 DEG C. and reaches a temperature above about 120 DEG C. in less than about 1% of the total time the biomass material is held at the temperature above about 120 DEG C.; a reduced recalcitrance material is initially at a temperature below about 110 DEG C. and reaches a temperature above about 120 DEG C. in less than about 20% of the total time the biomass material is held at the temperature above about 120 DEG C.; a reduced recalcitrance material is initially at a temperature below about 110 DEG C. and reaches a temperature above about 120 DEG C. in less than about 10% of the total time the biomass material is held at the temperature above about 120 DEG C.; a reduced recalcitrance material is initially at a temperature below about 110 DEG C. and reaches a temperature above about 120 DEG C. in less than about 5% of the total time the biomass material is held at the temperature above about 120 DEG C.; a reduced recalcitrance material is initially at a temperature below about 110 DEG C. and reaches a temperature above about 120 DEG C. in less than about 1% of the total time the biomass material is held at the temperature above about 120 DEG C.; a time for a biomass that is at a temperature below about 50 DEG C. to reach a temperature above about 120 DEG C. is less than about 6 min; a time for a biomass that is at a temperature below about 50 DEG C. to reach a temperature above about 120 DEG C. is less than about 3 min; a time for a biomass that is at a temperature below about 50 DEG C. to reach a temperature above about 120 DEG C. is less than about 1 min; a time for a biomass that is at a temperature below about 50 DEG C. to reach a temperature above about 120 DEG C. is less than about 30 sec; a time for a biomass that is at a temperature below about 50 DEG C. to reach a temperature above about 120 DEG C. is less than about 10 sec; a time for a biomass that is at a temperature below about 70 DEG C. to reach a temperature above about 120 DEG C. is less than about 6 min; a time for a biomass that is at a temperature below about 70 DEG C. to reach a temperature above about 120 DEG C. is less than about 3 min; a time for a biomass that is at a temperature below about 70 DEG C. to reach a temperature above about 120 DEG C. is less than about 1 min; a time for a biomass that is at a temperature below about 70 DEG C. to reach a temperature above about 120 DEG C. is less than about 30 sec; a time for a biomass that is at a temperature below about 70 DEG C. to reach a temperature above about 120 DEG C. is less than about 10 sec; a time for a biomass that is at a temperature below about 90 DEG C. to reach a temperature above about 120 DEG C. is less than about 6 min; a time for a biomass that is at a temperature below about 90 DEG C. to reach a temperature above about 120 DEG C. is less than about 3 min; a time for a biomass that is at a temperature below about 90 DEG C. to reach a temperature above about 120 DEG C. is less than about 1 min; a time for a biomass that is at a temperature below about 90 DEG C. to reach a temperature above about 120 DEG C. is less than about 30 sec; a time for a biomass that is at a temperature below about 90 DEG C. to reach a temperature above about 120 DEG C. is less than about 10 sec; a time for a biomass that is at a temperature below about 110 DEG C. to reach a temperature above about 120 DEG C. is less than about 6 min; a time for a biomass that is at a temperature below about 110 DEG C. to reach a temperature above about 120 DEG C. is less than about 3 min; a time for a biomass that is at a temperature below about 110 DEG C. to reach a temperature above about 120 DEG C. is less than about 1 min; a time for a biomass that is at a temperature below about 110 DEG C. to reach a temperature above about 120 DEG C. is less than about 30 sec; a time for a biomass that is at a temperature below about 110 DEG C. to reach a temperature above about 120 DEG C. is less than about 10 sec; heating a slurry includes heating by steam injection heating; heating a slurry includes heating by externally modulated steam injection heating; heating a slurry includes heating by internally injected steam injection heating; heating includes heating a slurry in a tube reactor; heating includes heating a slurry in a static tube reactor; heating includes heating a slurry in a tube reactor configured as a heated screw conveyor; heating includes heating a slurry in a tube reactor configured as a pressure cooker; heating includes heating a slurry utilizing indirect heating; heating includes heating a slurry utilizing a heated screw conveyor; heating includes heating a slurry utilizing a heated pressure cooker; heating includes heating a slurry in a tube reactor while agitating the slurry; agitating a slurry comprises mixing the slurry with a mechanical mixer; agitating a slurry comprises mixing the slurry with an auger mixer; agitating a slurry comprises mixing the slurry with a jet mixer; agitating a slurry comprises mixing the slurry with a recirculating pump; agitating a slurry comprises mixing the slurry with a progressive cavity pump; material is cooled utilizing a flash tank; cooling a material utilizing a flash tank to a temperature between about 50 and 110 DEG C.; cooling a material utilizing a flash tank to a temperature between about 90 and 110 DEG C.; cooling a material utilizing a cooling fluid fed heat exchanger; cooling a material utilizing a cooling fluid fed heat exchanger to a temperature between about 20 and about 110 DEG. C.; cooling a material utilizing a cooling fluid fed heat exchanger to a temperature between about 20 and about 80 DEG. C.; cooling a material utilizing a cooling fluid fed heat exchanger to a temperature between about 30 and about 70 DEG C.; a material is saccharified; a material is saccharified utilizing an enzyme; a material is contacted with an enzyme or organism; a lignocellulosic material utilized is wood; a lignocellulosic material utilized is particle board; a lignocellulosic material utilized is forestry wastes; a lignocellulosic material that is utilized is sawdust; a lignocellulosic material that is utilized is aspen wood; a lignocellulosic material that is utilized is wood chips; a lignocellulosic material that is utilized is grasses; a lignocellulosic material that is utilized is switchgrass; a lignocellulosic material that is utilized is miscanthus; a lignocellulosic material that is utilized is cord grass; a lignocellulosic material that is utilized is reed canary grass; a lignocellulosic material that is utilized is grain residues; a lignocellulosic material that is utilized is rice hulls; a lignocellulosic material that is utilized is oat hulls; a lignocellulosic material that is utilized is wheat chaff; a lignocellulosic material that is utilized is barley hulls; a lignocellulosic material that is utilized is agricultural waste; a lignocellulosic material that is utilized is silage; a lignocellulosic material that is utilized is canola straw; a lignocellulosic material that is utilized is wheat straw; a lignocellulosic material that is utilized is barley straw; a lignocellulosic material that is utilized is oat straw; a lignocellulosic material that is utilized is rice straw; a lignocellulosic material that is utilized is jute; a lignocellulosic material that is utilized is hemp; a lignocellulosic material that is utilized is flax; a lignocellulosic material that is utilized is bamboo; a lignocellulosic material that is utilized is sisal; a lignocellulosic material that is utilized is abaca; a lignocellulosic material that is utilized is corn cobs; a lignocellulosic material that is utilized is corn stover; a lignocellulosic material that is utilized is soybean stover; a lignocellulosic material that is utilized is corn fiber; a lignocellulosic material that is utilized is alfalfa; a lignocellulosic material that is utilized is hay; a lignocellulosic material that is utilized is coconut hair; a lignocellulosic material that is utilized is sugar processing residues; a lignocellulosic material that is utilized is bagasse; a lignocellulosic material that is utilized is beet pulp; a lignocellulosic material that is utilized is agave bagasse; a lignocellulosic material that is utilized is algae; a lignocellulosic material that is utilized is seaweed; a lignocellulosic material that is utilized is manure; a lignocellulosic material that is utilized is sewage; a lignocellulosic material is fermented and then heated a second time to a temperature greater than about 120 DEG C. for a time sufficient to further reduce the recalcitrance of the material; a lignocellulosic material is heated at a rate of between about 340 DEG C.·Kg/min and about 10,000,000 DEG C.·Kg/min; a lignocellulosic material is heated at a rate of between about 340 DEG C.·Kg/min and about 100,000 and about 500,000 DEG C.·Kg/min; a reduced recalcitrance material has an average particle size between about 0.25 mm and about 3 mm; a reduced recalcitrance material has an average particle size between about 0.5 mm and about 2 mm.

Features, for example, can also include: a method for processing a biomass material; heating a reduced recalcitrance lignocellulosic material to a temperature greater than about 120 DEG C. using steam and forming a slurry by combining the steam and the biomass material wherein the steam condenses and wets the biomass by condensation onto the biomass; heating a reduced recalcitrance lignocellulosic material to a temperature greater than about 120 DEG C. using steam and forming a slurry by combining the steam and the biomass material wherein the steam condenses and wets the biomass by condensation into the interior surfaces of the biomass; heating a reduced recalcitrance lignocellulosic material to a temperature greater than about 120 DEG C. using steam and forming a slurry by combining the steam and the biomass material wherein the steam condenses and wets the biomass by condensation onto the exterior surfaces of the biomass; a lignocellulosic material is heated at a rate of between about 340 DEG C.·Kg/min and about 10,000,000 DEG C.·Kg/min; a lignocellulosic material is heated at a rate of between about 100,000 and about 500,000 DEG C.·Kg/min; a slurry is held at the temperature greater than about 120 DEG C. for at least 1 min; a slurry is held at the temperature greater than about 120 DEG C. for at least 5 min; a slurry is held at the temperature greater than about 120 DEG C. for at least 10 min; a slurry is held at the temperature greater than about 120 DEG C. for at least 20 min; a slurry is held at the temperature greater than about 120 DEG C. for at least 30 min; a slurry is held at the temperature greater than about 120 DEG C. for at least 1 hour; a slurry is held at the temperature greater than about 120 DEG C. for at least 4 hours; a slurry is held at the temperature greater than about 120 DEG C. for at least 8 hours; a slurry is held at the temperature greater than about 120 DEG C. for at least 12 hours; a biomass comprises a slurry of at least about 10 wt % solids; a biomass comprises a slurry of at least about 20 wt % solids; a biomass comprises a slurry of at least about 30 wt % solids; a biomass comprises a slurry of at least about 40 wt % solids; a biomass comprises a slurry of at least about 50 wt % solids; cooling a slurry to a temperature between about 20 and about 80 DEG C.; cooling a slurry to a temperature between about 30 and about 70 DEG C.; steam and biomass material are combined utilizing a Jet Cooker; a slurry is collected in a tube reactor that is agitated internally utilizing a mechanical agitator; a slurry is collected in a tube reactor that is agitated internally utilizing an auger mixer; a slurry is collected in a tube reactor that is agitated internally utilizing a jet mixer; a slurry is collected in a tube reactor that is agitated internally utilizing a recirculating pump; contacting a biomass with an organism or enzyme; a reduced recalcitrance material is made by irradiating a cellulosic or lignocellulosic material with between about 10 and 50 Mrad of ionizing radiation; a reduced recalcitrance material is made by irradiating a cellulosic or lignocellulosic material with between about 20 and 40 Mrad of ionizing radiation; producing a sugar solution by enzymatically saccharifying a cellulosic or lignocellulosic material; the recalcitrance of a cellulosic or lignocellulosic material has been reduced by irradiating the material and steam heating the material to a target temperature; the recalcitrance of a cellulosic or lignocellulosic material has been reduced by irradiating the material and steam heating the material to a target temperature for between about 1 and about 240 min; the recalcitrance of a cellulosic or lignocellulosic material has been reduced by irradiating the material with between about 10 and about 50 Mrad or radiation and steam heating the material to a target temperature; the recalcitrance of a cellulosic or lignocellulosic material has been reduced by irradiating the material and steam heating the material to a target temperature between about 120 and 160 DEG C.; a sugar that is produced is fermented.

Other features and advantages of the invention will be apparent from the following detailed description, and from the claims.

### DESCRIPTION OF THE DRAWING

The foregoing will be apparent from the following more particular description of example embodiments of the invention, as illustrated in the accompanying drawings. The drawings are not necessarily to scale, emphasis instead being placed upon illustrating embodiments of the present invention.

FIG. 1 is a flow diagram showing processes for manufacturing sugar solutions and products derived therefrom.

FIG. 2 is a flow diagram showing the steps that can be used to implement heating of a feedstock to reduce its recalcitrance.

FIG. 3 shows a schematic of a possible embodiment of the invention.

FIG. 4 shows a cross cut view of a direct steam heater that is externally modulated useful for rapidly heating a biomass containing slurry.

FIG. 5 shows a cross cut view of a direct steam heater that is internally modulated useful for rapidly heating a biomass.

FIG. 6 shows a cross cut view of a Jet Cooker type direct steam injector that is coupled to and feeds directly to a tube reactor.

FIG. 7 shows diagrammatically plant components for processing a biomass material.

FIG. 8 is a highly diagrammatic depiction of the possible effect of the processing described herein on a lignocellulosic material.

### DETAILED DESCRIPTION

Using the equipment, methods and systems described herein, cellulosic and lignocellulosic feedstock materials, for example that can be sourced from biomass (e.g., plant biomass, animal biomass, paper, and municipal waste biomass), can be turned into useful products and intermediates such as sugars and other products (e.g., fermentation products). Included are equipment, methods and systems to reduce or further reduce the recalcitrance of these materials.

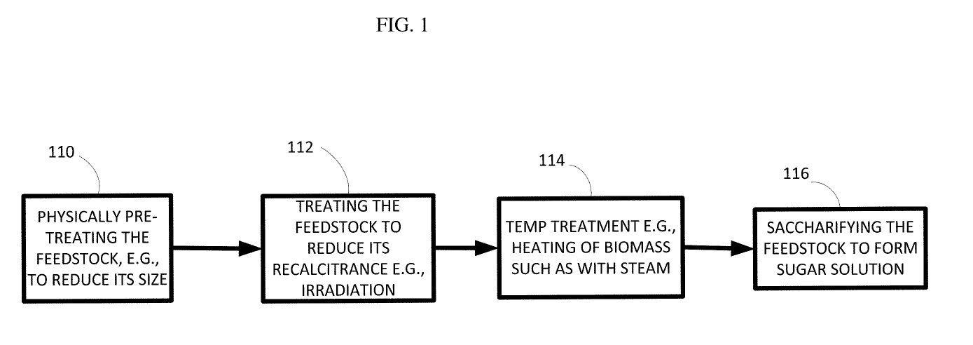

Referring to FIG. 1, processes for manufacturing sugar solutions and products derived therefrom include, for example, optionally mechanically treating a cellulosic and/or lignocellulosic feedstock 110. Mechanical treatments can, e.g., reduce the size of the biomass and/or reduce the recalcitrance of the biomass. Before and/or after this treatment, the feedstock can be treated with another physical, mechanical and/or chemical treatment, for example irradiation, to reduce, or further reduce its recalcitrance 112. After such treatments, the material can be heated 114 to a target temperature such as above about 90 (e.g., between about 90 and about 200 DEG. C.), e.g., for a time sufficient to further reduce the recalcitrance of the material, e.g., between about 1 min and 240 min. The material that is heated in step 114 can be, for example a biomass material, e.g., a particulate biomass (e.g., a loose material, a flowable particulate biomass). The biomass can be suspended in a fluid (e.g., water), providing a slurry or it can be a dry biomass, such as having less than 25 w.t. % moisture, less than about 15 w.t. % moisture, less than about 10 w.t. % moisture or even bone dry such as having less than about 5 w.t. % moisture. The biomass can also be a wet biomass. After the recalcitrance reduction steps (e.g., any one or more of steps 110, 112 and 114 in any order and optionally repeated one or more times), a sugar solution can be formed by saccharifying the feedstock 116 by, for example, the addition of one or more enzymes or an acid. A product can be derived from the sugar solution, for example, by fermentation to an alcohol or an acid, such as lactic acid. Further processing can include purifying the solution, for example by filtering and distillation.

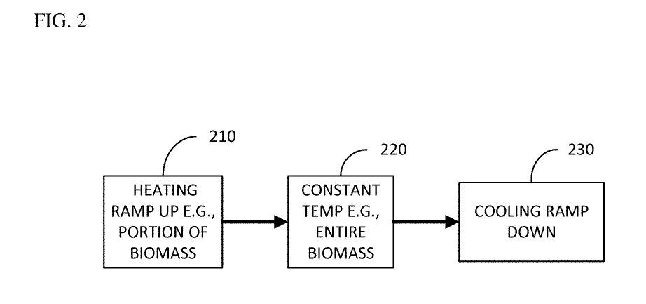

FIG. 2 is a flow diagram showing the steps that can be used to implement heating of the feedstock in 114. In particular, the feedstock, such as a reduced recalcitrance lignocellulosic or cellulosic material (e.g., ionizing radiation treated), is heated to a temperature 210 by ramping the temperature at a ramp rate. All the material can be heated together, or portions of the biomass can be heated. For example, portions can be flowed to or through a heater and heated. For example, the material (e.g., biomass material such as dry particulate biomass or a slurry of biomass) can flow at a rate of between about 1 gpm and about 15,000 gpm and if it is heated to between about 90 and about 170 DEG C. above its initial temperature, the temperature increase can be between about 340 DEG C.·Kg/min and about 10,000,000 DEG C.·Kg/min (e.g., between about 100,000 and about 500,000 DEG C.·Kg/min). The rate of heat increase is preferably maximized since this can impact the overall processing rate and can also improve the recalcitrance reduction. The target temperature can be between about 90 and 200 DEG C. (e.g., about 90, 95, 99, 100, 105, 107, 110, 112, 115, 118, 125, 132, 136, 138, 140, 145, 150, 160, 163, 165, 168, 170, 175, 180, 185, 190, 195). For example the temperatures can be chosen to be between about 100 and about 200 DEG C., between about 100 and about 180 DEG C., between about 120 and about 200 DEG C., between about 120 and about 180 DEG C., between about 120 and about 160 DEG C., between about 120 and about 140 DEG C., between about 140 and about 200 DEG C., between about 140 and about 180 DEG C., between about 140 and about 160, between about 140 and about 180 DEG C.

The amount of time for the heat treatment 220 can be between about 1 min and about 240 min (e.g., about 1, 2, 3, 4, 5, 10, 12, 15, 18, 22, 25, 30 35, 39, 45, 60, 120, 140, 150, 160, 170, 180, 190, 200, 210, 220, 230 or 240 min). For example, the material can be heated at about 95 DEG C. for about 60 to 240 min, about 100 DEG C. for about 60-200 min, about 105 DEG C. for about 60-100 min, about 110 DEG C. for about 50-90 min, about 120 DEG C. for about 40-80 min, 140 DEG C. for about 20-70 min, 150 DEG C. for about 10-60 min, about 150 DEG C. for about 5-20 min, about 160 DEG C. for about 1-5 min. The amount of time for the treatment can depend on many different factors such as the degree of recalcitrance of the feedstock, the feedstock composition (e.g., the amount of lignin, cellulose, hemicellulose, other components such as starch and inorganic materials), the kind and amount of other physical treatments (e.g., such as the amount and type of comminution, the amount and type of irradiation, the surface area, the porosity) and the temperature of the heat treatment. For example, heating of material at 140 DEG C. can take between about 1 and 6 hours (e.g., between 3 and 5 hours, about 4 hours) to reduce the recalcitrance sufficiently, while heating of material at 160 DEG C. can take between about 15 min and about 1 hour (e.g., between about 20 min and about 50 min, about 30 min) to reduce the recalcitrance to about the same level. Heating above about 200 DEG C. can produce a high degree of recalcitrance reduction but can also degrade the material, therefore reducing the final yield of sugars after the saccharification step. Such degradation can also hinder other processes, such as fermentation, e.g., because of toxic materials that can be produced, e.g., furfural. Irradiation below about 20 Mrad can require a longer heat treatment (e.g., 2 times longer, 3 or more times longer) to achieve the same amount of saccharification in a subsequent step, than irradiation at between about 20 and 40 Mrad. If the material in step 210 is heated in portions, the material in step 220 can be collected and heated as a batch (e.g., in a pressure cooker, mixed tube reactor, static tube reactor) or the material in step 220 can be heated continuously (e.g., using a continuous tube reactor). After the heating treatment 220, the material is cooled 230. Cooling can be passive, for example by allowing the material to thermally equilibrate with room temperature, or the cooling can be done by contacting the biomass with a heat exchanger. Such heat can be utilized to generate energy (e.g., by providing the heat to a boiler utilized in a steam turbine) or transferred to other processes that may require heat (e.g., saccharification, drying, fermentation). Cooling can also be done by flashing the material, e.g., into a flash tank.

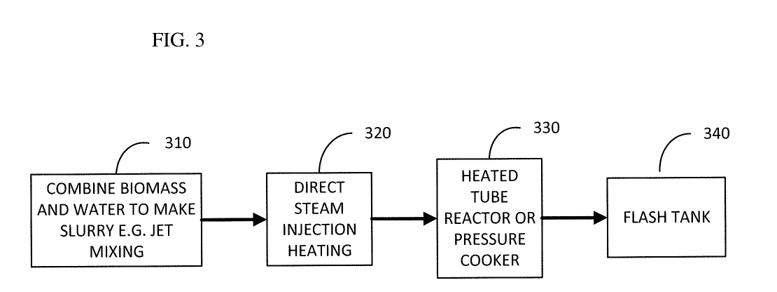

FIG. 3 shows a schematic of a possible embodiment of the invention. A biomass (e.g., lignocellulosic material or cellulosic material that has been irradiated) can be combined (e.g., mixed, dispersed) with a fluid such as water. For example the biomass can be dispersed in a tank equipped with mixers such as auger mixers and/or jet mixers 310. Optionally the material can be wet ground in this same tank to reduce the particulate size. This initial treatment can provide a slurry where the particles of biomass are homogeneously dispersed throughout the slurry at least while being mixed (e.g., wherein from top to bottom of the dispersing tank the difference in wt % of the biomass in a 1 cm³ volume is less than 50%, less than about 20%, less than about 10%, less than about 5% such as where the top is measured 1 cm below the average surface of the tank and the bottom is measured 1 cm above the average bottom of the tank). The slurry is then heated rapidly (e.g., at rates as previously described) utilizing continuous direct steam injection 320. Processes and equipment for direct steam injection will be discussed further below. After the direct steam injection, the material is maintained at the target temperature by having it flow or be conveyed through a heated continuous tube reactor/heated jacketed pressure cooker 330 (e.g., the tube reactor and pressure cooker can be considered equivalent). After the heating in a tube or pressure cooker, the slurry can be cooled using a flash tank 340. Alternatives for cooling include or further include cooling using a cooled tube reactor or cooling using cooling fluid in the pressure cooker(e.g., the pressure cooker can be configured as a tube reactor).

The pressure cooker or tube reactor can include mixers such as auger mixers, jet mixers, progressive cavity pumps (e.g., provided or modified from Seepex Inc., Enon Ohio), recirculating loops and combinations of these to ensure the biomass material does not settle and that there is efficient energy transfer with the heating apparatus. For example heating can be provided by a heating jacket. Tube reactors/pressure cookers can be advantageous because their cost can be low, but other types of reactors can be utilized. Tube reactors can be configured, for example, as comprising a tube with length to width (e.g., an aspect ratio) of at least about 1 (e.g., at least 2, at least 3, at least 4, at least 5, at least 6, at least 7, at least 8, at least 9, at least 10).

Generally, two types of heat exchangers, direct and indirect, are used to transfer heat between process materials (e.g., dry particulate biomass, slurried biomass) and heating/cooling fluids (e.g., water, oils, steam). Direct heat transfer occurs by direct contact of the heating medium with the process material, such as a slurry including biomass. For indirect heating, the process material and heating medium are separated by a thermally conductive barrier so that heat is exchanged by the materials but the heating medium and process fluid/slurry are never in contact.

Generally, indirect-contact heat exchangers, such as shell-and-tube, plate-and-frame, or scraped surface exchangers, have two or more fluid flow paths that do not allow for direct mixing of the fluids. They promote the transfer of heat from one fluid to another across a thermally conducting, but otherwise impermeable, barrier such as a tube wall or plate. For example, a heating jacket on a tube reactor or a pressure cooker exemplifies an indirect heat exchanger. Indirect heating is beneficial in avoiding mixing of the cooling or heating fluid with the fluid to be heated. This can be important if the fluid to be processed cannot tolerate this mixing, e.g., should not be diluted or there are risks in contamination such as by an organism.

An example of a direct-contact heat exchanger is addition of ice into a reaction vessel. Another example of a direct-contact heat exchanger is the injection of metered amounts of steam into a process fluid such as a liquid or slurry that needs to be heated. Injecting steam directly into the process fluid/slurry results in more rapid heat transfer and more efficient energy usage than indirect heat exchangers. Direct-contact steam heating can provide up to almost 100% thermal efficiency, because both the sensible and the latent heat of the steam are used. This transfer of energy is maximized if the steam is made to condense into the material. Direct contact steam heaters can be classified as externally modulated or internally modulated. For externally modulated heaters, steam flow control into a flowing material (e.g., a slurry, liquid or flowing particulate material) controls the temperature and pressure between the steam and liquid. Efficient and rapid transfer of the thermal energy is achieved. In some configurations, accurate temperature control can be difficult to maintain under varying load conditions. Some examples of externally modulated heaters including tank spargers, in-line spargers, and mixing tees.

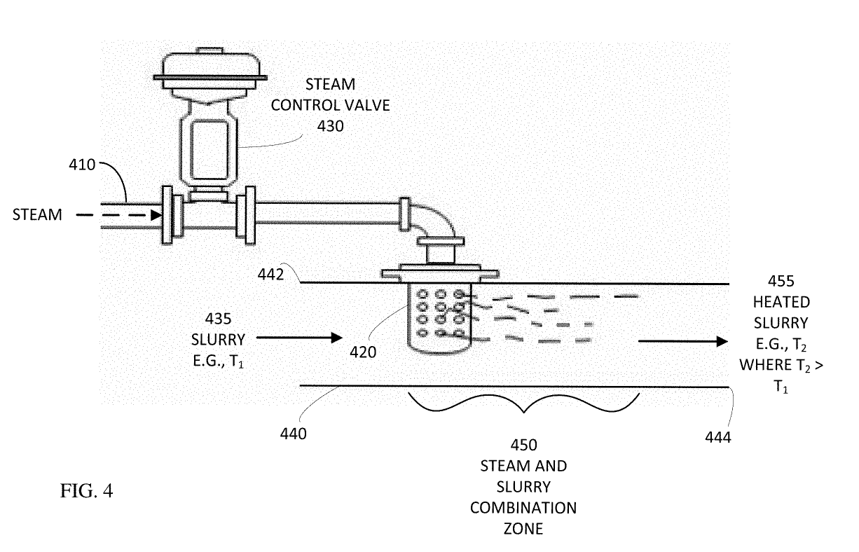

FIG. 4 shows a cross cut view of a direct steam heater that is externally modulated and useful for rapidly heating a biomass containing slurry. The heater is configured as an in line sparger where steam is fed through a tube 410 to the steam diffuser 420 and controlled by the steam control valve 430. Slurry 435 enters the heater 440 through an opening upstream from the diffuser, and is made to flow past the steam diffuser mounted into the heater. The heater can be a pipe or section of a pipe with a slurry inlet at an upstream end 442 and a downstream slurry outlet end 444. Within the heater there is a heating zone 450 where the steam and slurry combine. The turbulent flow of the slurry and injection of steam aids in mixing the steam throughout the slurry as it flows, thus heating the slurry uniformly. The turbulent flow can be increased by adding some physical barriers such as dimples in the inner surface of the heater downstream from the steam diffuser, or otherwise providing a tortuous path for the flow of slurry, although these modifications can impede the flow of material. In addition, since steam is dispersed through the many holes located in the diffuser, it contacts a large volume of the slurry as it flows past and heat can be quickly distributed throughout the flowing slurry. In addition to the physical design of the heater, the heating zone can vary depending on several other factors such as flow rates, material composition, steam temperature, steam pressure and initial temperature. Generally, the heating zone can be considered to start at the steam diffuser and end at a point downstream where the heat throughout the slurry is uniform and at the target value. The slurry can be heated quickly to a targeted temperature by controlling the vapor flow and temperature using this externally modulated steam injection system. Heated slurry 455 exits the heater or heating zone and can be fed to a heated pressure cooker or heated tube reactor as previously described. In alternate embodiments, the heater can be utilized for heating a particulate biomass feedstock, e.g., conveyed into the heater pneumatically.

An internally modulated heater controls both steam flow and mixing by employing a stem/plug assembly inside the heater. Controlling the position of the stem/plug controls the steam discharge area of the nozzle. This, in turn, controls the amount of steam that is allowed to pass through the nozzle. Internal modulation eliminates the need for an external steam control valve. Internally modulated direct contact steam heaters inject metered amounts of steam into the process fluid through a variable area steam nozzle. The nozzle design ensures constant steam pressure and velocity at the point where steam contacts the liquid or slurry, eliminating the potential for pressure upsets and ensuring smooth heater operation. Internally modulated direct-contact steam heaters are cleaned by their own turbulent mixing action, so they do not encounter fouling or scale buildup. They also have the flexibility to heat slurries containing a high concentration of solids or non-Newtonian liquids. Solid materials (e.g., powders, particulate material, loose fibrous material) can also be heated, for example, being conveyed into the heater by venturi effect and collected downstream as a heated slurry.

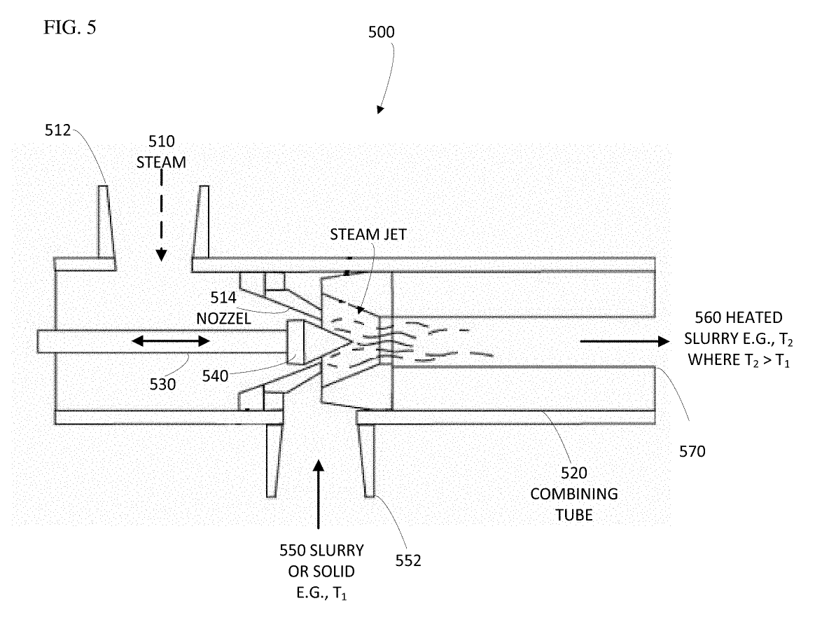

FIG. 5 shows a cross cut view of a direct steam heater 500 that is internally modulated and useful for rapidly heating a biomass. The feedstock can be a solid, for example a solid particulate cellulosic or lignocellulosic material (e.g., powder, particulates, loose fibers). The feedstock can also be a slurry, for example a combination of a solid biomass with a fluid (e.g., water). This kind of heat exchanger is also known as a Jet Cooker. Steam 510 enters the heater at full pressure through an opening 512 and moves to the steam nozzle 514 which opens to the combining tube 520. The flow of the steam is controlled by the stem 530 and plug 540 mounted thereupon. For example the stem can move in the direction indicated by the double headed arrow (e.g., closed to the right, open to the left). Feedstock (e.g., solids or slurry) 550 enters the heater through opening 552 where it is contacted with a steam jet (created by the nozzle and plug) as it moves into the combining tube 520. In a first instance, the material would be blown into the pipe by the steam (e.g., venturi effect). In some optional embodiments, several openings could be utilized. The turbulent flow of the feedstock and steam rapidly mixes the feedstock. The steam transfers heat throughout the feedstock as it moves down the combining tube. Heated slurry 560 exits the heater through opening 570 and can be fed to a heated tank or heated tube reactor as previously described.

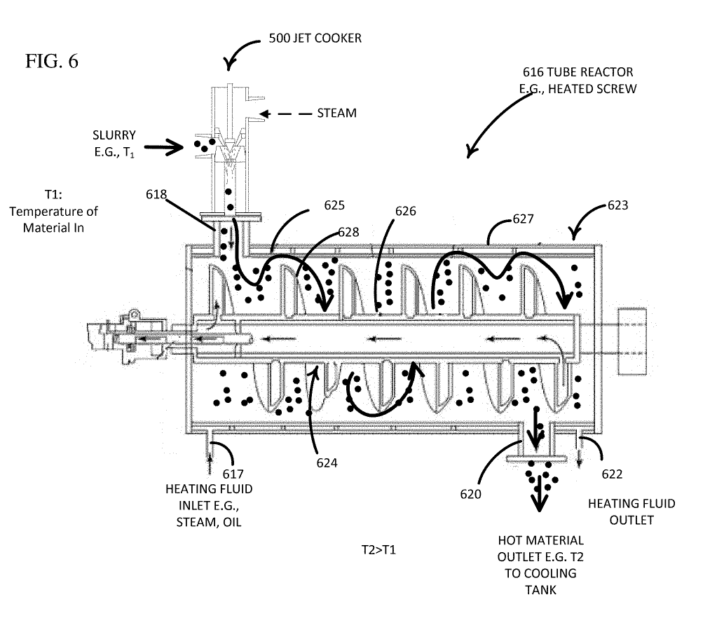

As described previously, implementations of the invention include coupling rapidly heating a feedstock and then maintaining temperature of a heated slurry for a desired time. For example FIG. 6 shows a cross cut view of a Jet Cooker type direct steam injector that is coupled to and feeds directly to a tube reactor. The Jet Cooker 500 has already been described. The tube reactor is configured as a heated screw conveyor. Heating fluid such as steam, pressurized water or oil flows through a casing 623, which includes an inner shell 625 and an outer shell 627 that define a space therebetween for fluid flow. Feedstock (e.g., slurry) that has been heated by Jet Cooker 500 is conveyed through the tube reactor 616 (e.g. heated screw cooler) by a conveyor that comprises a rotating screw 624. The shaft 626 and flight 628 of the screw 624 are hollow, and are also heated by a heating fluid (e.g., steam, oil, pressurized water). A drive motor (not shown) is mechanically connected to the shaft providing the torque needed to rotate the shaft and screw. In 616, the biomass slurry is subjected to continuous movement by the helices of the screw 625 and is constantly mixed. The rotation, conveying, interfolding and dispersing action increases the heat exchange between the slurry biomass and the heating fluids in the screw and casing as well as keeping thick slurries moving and avoiding separation of the liquids and solids (e.g., settling is avoided). The heating fluid can be set at the target temperature for the treatment. Optionally, the target temperatures of the Jet Cooker and the heated screw conveyor can be different. For example the target temperature of the Jet Cooker can be higher (e.g., at least about 5 DEG C. higher, at least about 10 DEG C. higher, at least about 20 DEG higher, at least about 50 DEG C. higher) than the target temperature of the heated screw conveyor. Alternatively, the target temperature of the Jet Cooker can be lower than the target temperature of the heated screw conveyor (e.g., at least about 5 DEG C. lower, at least about 10 DEG C. lower, at least about 20 DEG C. lower, at least about 50 DEG C. lower). The screw conveyor can also further grind and comminute the biomass in the slurry. In some embodiments the screw conveyor can include multiple screws (e.g., a dual screw conveyor). In other embodiments, the screw conveyor is configured or used as a batch reactor. For example, the auger can be configured or used to mix the material throughout the tube e.g., by conveying material in both directions along the tube as it is filled through 618. Meanwhile, the material can be heated by the heat exchangers as described. Once the required heating is accomplished the material can be conveyed out through 620.

The hot slurry exiting at 620 (or any heated slurry after a desired heating time has elapsed) is subsequently cooled. Ideally the material is cooled to the optimal temperature for a subsequent processing step, e.g., saccharification. Cooling can be done by using a flash tank as previously described. Cooling can also be done by adding the heated slurry to cooled water, although this will dilute the slurry and may impose a cost burden to remove the water further downstream in the processing of the material. In some preferred embodiments, the material is cooled by a heat exchanger and the heat is reused. For example, the material can be cooled to the optimal temperature of saccharification (e.g., between about 40 and 60 DEG C. for enzymatic saccharification as described below), where the cooling is done using a heat exchanger such as a cooled screw conveyor or a cooling jacket. Cooling fluids that have been heated up during the cooling in the heat exchanger can also be utilized to help maintain the temperature of the saccharification at the optimal temperature or can be used in any other process (e.g., fermentation, heating of the facility, pre-heating boiler water for the steam generator). Combinations of cooling methods can also be used (e.g., flash tank, cooling water, heat exchangers in any order, combination and optionally used repeatedly).

Steam heaters can be purchased, and/or modified from commercial steam heaters. For example, some commercial products are available from Pick Heaters inc. (West Bend, Wis.), Hydro Thermal Inc. (Waukesha, Wis.), ProSonix LLC (Milwaukee, Wis.), Kandant Inc. (Three Rivers, Mich.), Komax Systems Inc. (Huntington Beach, Calif.) and Spirax-Sarco Ltd (Blythewood, S.C.).

In some embodiments, several steam heaters can be used, for example in series. For example, two or more Jet Cookers. Utilizing such a configuration can be advantageous because the total heating and/or amount of material can be modulated (e.g., the water amount, biomass material and temperatures can be controlled more easily and/or accurately). In addition, the jet cookers can be disposed along the length of a pipe and inject pressurized steam into the pipe so as to help in the processing of the material, e.g., by helping to convey the material, heat the material, dilute the material or combinations of these.

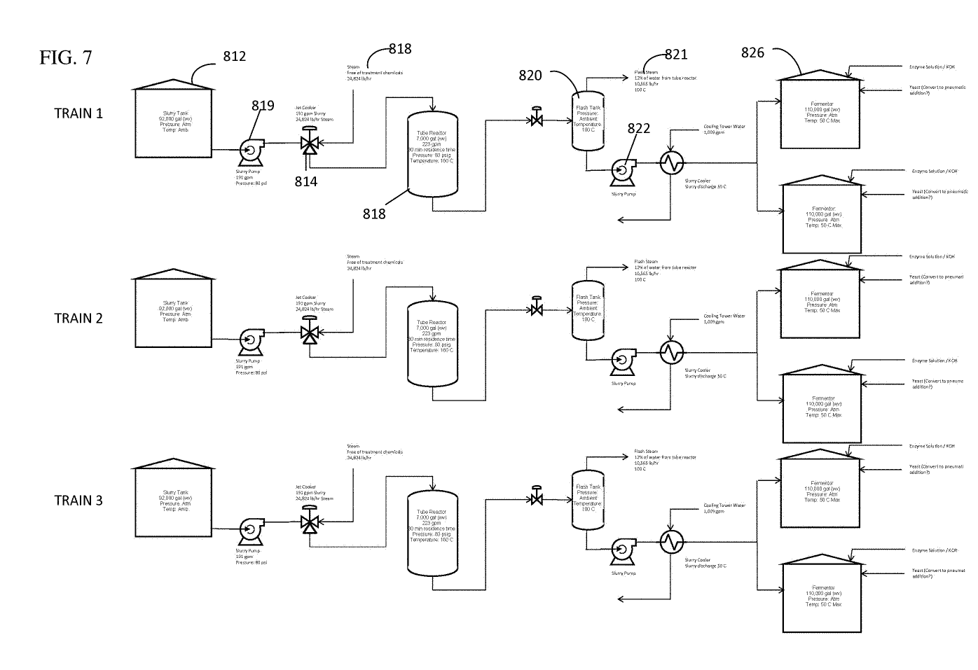

FIG. 7 shows diagrammatically another possible embodiment, for example to make products from a biomass. In particular, the figure shows how three trains of processing equipment can be utilized to process materials (e.g., train 1, train 2 and train 3). Optionally more (e.g., more than 3, more than 4, more than 5, or even more than 6) trains can be utilized. Optionally fewer than 3 trains can be utilized (e.g., one or two). Increasing the trains allows higher throughput of the material. Each train consists of a slurry tank 812 (e.g., 92, 000 gal, atm pressure, ambient temp), a slurry pump 819 (e.g., 191 gpm, 80 psi), a Jet Cooker 814 (e.g., 191 gpm, 24, 824 lb/hr steam), a steam free of treatment chemicals 816, a tube reactor 818 (e.g., 700 gal, 80 min residence time, 80 PSIG, 160 DEG C.), a flash tank 820 (e.g., ambient pressure, 100 DEG C.) that can flash steam 821 (e.g., 12% of the water from the tube reactor, 10565 lb/hr, 100 DEG C.), a slurry pump 822, a heat exchanger 824 cooled utilizing cooling tower water 826 (e.g., 1009 gpm), two fermenters (e.g., for saccharification using enzymes/KOH and/or fermentation using Yeast, 110,00 gal, atmospheric pressure, max temp 50 DEG C.). Different components can be utilized in the train. For example, the tank 812 can be replaced with a hopper to contain dry biomass that is fed to the Jet Cooker, and/or the tube reactor 818 can include mixers (e.g., jet mixers) or recirculating pumps to ensure the slurry therein is well dispersed (e.g., solids do not settle).

Materials can be conveyed by various means between (e.g., from and to) various components (e.g., slurry tank, Jet cookers, tube reactors, flash tanks, fermenters). For example, pumps such as progressive cavity pumps with ribbon augers can be utilized to move the material. In addition augers or screw conveyors can be utilized, for example, in pipes. In some optional embodiments the distances between the various components is minimized to avoid conveying materials that may be difficult to pump or otherwise convey long distances.

Steam that can provide the heating for the various heat exchangers described herein can be produced by co generation, for example as described in PCT/US14/21634 filed Mar. 7, 2014, the entire disclosure of which is herein incorporated by reference. The steam can be heated to any desired temperature. For example, boilers can produce super-heated steam (e.g., 500 PSIG and 650 DEG F.). Lower pressures and lower temperature steam can also be used, for example, steam drawn off as “cooled” steam from a turbine (e.g., 80 PSIG and 310 DEG F.). For example, for externally modulated and internally modulated steam used with the direct steam heat exchangers the steam temperature can be between about 350 DEG C. and about 120 DEG C. (e.g., between about 250 and 140 DEG C.) and the steam pressure can be between about 500 PSIG and about 50 PSIG (e.g., between about 400 PSIG and about 80 PSIG, between about 300 PSIG and about 100 PSIG, between about 200 and about 100 PSIG). The rate of steam supplied can be at least about 10,000 lb/hr (e.g., at least 20 kLb/hr, at least 50 kLb/hr, at least 75 kLb/hr, at least about 100 kLb/hr). The steam that is utilized must be clean (e.g., not containing anti-corrosion additives or other additives) since these can interfere with downstream processing of the biomass (e.g., saccharification, fermentation) and/or add purification complexity to possible products (e.g., sugars, alcohols). For example, clean steam can be generated utilizing a boiler with stainless steel piping and construction to avoid corrosion.

Without being bound by a specific mechanism, it is believed that irradiation can be important in enabling the use of the slurries with the various heat exchangers herein described because the dipersability of the feedstock in the fluid to make a slurry and the flow properties of the slurry thus made appear to be dependent on the level of irradiation. For example, where it may be difficult to make a flowable and mixable slurry comprising more than about 10 wt % (e.g., more than 20 wt %) biomass that is not irradiated, it is possible to make higher solids slurries that flow (e.g., greater than about 20 wt %, greater than about 30 wt %, greater than about 40 wt % and even greater than about 50 wt %). Therefore, the irradiation as describe here e.g. wherein a dose between about 10 and 50 Mrad is utilized, can help in providing a flowable slurry that can be heated utilizing a direct steam injection.

Table 1 is an example showing the effect of irradiation, temperature and hold time on the Xylan and Glucose released from a particulate corn cob material.

| | | Irradiation | Xylan | Glucose |
|---|---|---|---|---|
| Temperature | Hold | Level | Released | release |
| (DEG C.) | Time | (Mrad) | (g/L) | (g/L) |
| 100 | 30 | 10 | 6 | 12 |
| 100 | 30 | 20 | 15 | 16 |
| 100 | 30 | 20 | 14 | 15 |
| 100 | 30 | 35 | 27 | 15 |
| 100 | 120 | 10 | 7 | 13 |
| 100 | 120 | 20 | 20 | 16 |
| 100 | 120 | 35 | 29 | 14 |
| 100 | 240 | 10 | 10 | 14 |
| 100 | 240 | 20 | 21 | 15 |
| 100 | 240 | 35 | 31 | 12 |
| 120 | 30 | 10 | 9 | 14 |
| 120 | 30 | 20 | 17 | 14 |
| 120 | 30 | 35 | 32 | 15 |
| 120 | 120 | 10 | 15 | 14 |
| 120 | 120 | 20 | 31 | 16 |
| 120 | 120 | 35 | 40 | 15 |
| 120 | 240 | 10 | 30 | 14 |
| 120 | 240 | 20 | 37 | 15 |
| 120 | 240 | 35 | 54 | 15 |
| 140 | 30 | 10 | 21 | 14 |
| 140 | 30 | 20 | 44 | 16 |
| 140 | 30 | 35 | 60 | 13 |
| 140 | 120 | 10 | 56 | 15 |
| 140 | 120 | 20 | 67 | 12 |
| 140 | 120 | 35 | 71 | 15 |
| 140 | 240 | 10 | 64 | 14 |
| 140 | 240 | 20 | 71 | 14 |
| 140 | 240 | 35 | 67 | 13 |
| 160 | 30 | 10 | 66 | 14 |
| 160 | 30 | 20 | 72 | 15 |
| 160 | 30 | 35 | 86 | 14 |
| 160 | 120 | 10 | 72 | 11 |
| 160 | 120 | 20 | 61 | 12 |
| 160 | 120 | 35 | 65 | 14 |
| 160 | 240 | 10 | 23 | 11 |
| 160 | 240 | 20 | 26 | 12 |
| 160 | 240 | 35 | 21 | 15 |

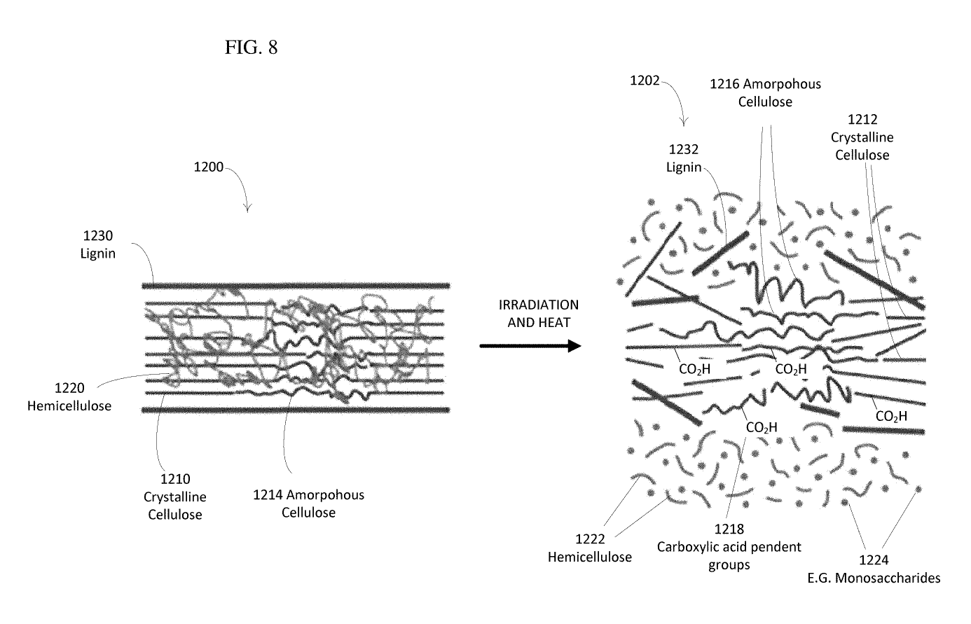

In addition, and again without being bound to a specific mechanism, the rapid heating rate and/or sustained heating of a slurry made from an irradiated biomass can provide a synergistic effect to reduce the recalcitrance of a lignocellulosic or cellulosic material and increase the amount of sugars released. FIG. 8 is a highly diagrammatic depiction of the possible effect of the processing described herein on a lignocellulosic material. A material 1200 is shown including crystalline cellulose 1210, amorphous cellulose 1214, hemicellulose 1220 and lignin 1230 components. Many of the methods described herein, such as irradiation followed by heat treatment in the presence of water, can provide cellulosic and/or lignocellulosic materials 1202 that have, for example, a lower recalcitrance level, a lower molecular weight, a different level of functionalization, a higher solubility (e.g., in water), a different level of crystallinity, an increased surface area, an increase in porosity, a different arrangement of components and/or a disrupted (e.g., broken, re-arranged) bonding between the components, relative to a native material or untreated material 1200. For, example, in comparison to the initial cellulose 1210, the cellulose components treated by the processes described herein have been changed to a lower molecular weight cellulose 1212 and can have a higher fraction of amorphous portions 1216, as well as being re-arranged with respect to lignin/hemicellulose components in the initial lignocellulosic material 1200. Some of the components can also be functionalized, for example with carboxylic acid pendent groups 1218 or other functional groups depending on quenching conditions (e.g., quenching an irradiated material in oxidizing conditions such as an oxygen environment will generally produce carboxylate groups). The hemicellulose is shown to have also undergone molecular weight reduction, for example to oligomers 1222 or even to monosaccharides 1224 (eg., xylose, arabinose). Furthermore, the close association of the hemicellulose 1220, crystalline cellulose 1210 and amorphous cellulose 1244 in the starting lignocellulosic material 1200 has been disrupted after treatments (e.g., irradiation followed by heating) where the cellulose derived materials (1212 and 1216) and hemicellulose derived materials (1222 and 1224) have been separated and bonding between the two can be disrupted/broken or rearranged. This separation can be particularly accelerated and facilitated by heating the slurry rapidly to high temperatures as described herein. The treatment by irradiation can increase the susceptibility to the swelling by the heated water because the radiation induced changes to the material (e.g., molecular weight reduction, functionalization, crystallinity reduction) make water penetration and surface activity much easier. Swelling by heat treatment prior to irradiation can be much less effective because the material is held together and can be more hydrophobic (e.g., more hydrophobic than irradiated and functionalized material). Irradiation can also be optimal utilizing a dried material, e.g., due to water absorbing at least a portion of the irradiation dose. Drying a swelled material can collapse the material back to the original un-expanded/un-swelled arrangement, especially if the material has not been previously treated (e.g., with irradiation). In addition to reduction of the molecular weight of hemicellulose and cellulose, the lignin molecular weight has been reduced 1232 in comparison to the starting lignin 1230 and the protective structure of the lignin has been disrupted allowing the close arrangement of the components in 1200 to be swelled to a more open and less organized structure in 1202. In some implementations, the recalcitrance reduction as described herein does not significantly release low molecular weight sugars such as mono and disaccharides because a hydrolysis step, such as enzymatic saccharification, is required (e.g., especially in the case of cellulose which is less susceptible to water hydrolysis than hemicellulose). For example only less than about 5% of the available sugars (e.g., available as cellulose or hemicellulose) can be released as mono saccharides by the methods. Rather, the recalcitrance reduction can cause molecular weight reduction (e.g., by irradiation). Contact with water then can swell the material e.g., by more than about 5 vol. % as compared to the unswelled dry material (e.g., a dry irradiated or non-irradiated material.) For example, the material can swell by at least about 10 vol. %, at least about 20 vol. %, at least about 30 vol. %, at least about 40 vol. %, at least about 50 vol. %. Heating the material as described herein (e.g., a slurry of irradiated biomass) greatly accelerates the swelling and/or swells the material to a higher degree, reducing the processing time and/or increasing the amounts of sugars released in the subsequent hydrolysis steps. The heat treatment can also decrease the crystallinity of the cellulosic material by, for example, dissolving parts of the crystalline cellulose. The biomass material can be a particulate material. For example, before the biomass material is swelled, it can have an average particle size above at least about 0.25 mm (e.g., at least about 0.5 mm, at least about 0.75 mm) and below about 6 mm (e.g., below about 3 mm, below about 2 mm). The biomass material can be a fibrous material, for example as described herein. In some embodiments this is produced by mechanical means, for example as described herein.

In addition to the above, without being bound by a specific mechanism, it is suggested the rapid heating rate of the slurry and/or particulate biomass can cause a rapid expansion of the various components in the biomass and the material cannot accommodate the expansion without some structural disruption (e.g., the relaxation time of the material to accommodate the additional thermal energy is shorter than the rate at which the material is heated up). The disruption can, for example, cause a reduction in the crystallinity of the material, reduction in molecular weight, and/or can microfracture the material.

The methods described herein e.g., that reduce the recalcitrance of lignocellulosic materials, can make the materials more readily utilized by a variety of microorganisms to produced useful produces. For example the methods can make the materials more susceptible to enzymes and biomass-destroying organisms that contain or manufacture various cellulolytic enzymes (e.g., cellulases, ligninases, xylanases and hemicellulases) or various small molecule biomass-destroying metabolites. For example cellulose is initially hydrolyzed by endoglucanases at random locations producing oligomeric intermediates, where the recalcitrance reduction of the lignocellulose can make the cellulosic components of the biomass more easily accessible to the biomass. Recalcitrance reduction can also improve the efficiency of other enzymes such as exo-splitting glucanases e.g., cellobiohydrolase, and cellobiose cleaving enzymes such as cellobiase. Recalcitrance reduction can improve the efficiency (e.g., effectiveness) of enzymatic processing of hemicellulose with xylanase (e.g., hemicellulase) which acts on this biopolymer to release xylose as one of the possible products. Swelling, such as caused by heating the slurries as described herein can optimize the access of the cellulose to the saccharifying enzymes.

### Radiation Treatment

As discussed above, the feedstock, such as a lignocellulosic or cellulosic material, can be treated with radiation to modify its structure to reduce its recalcitrance. Such treatment can, for example, reduce the average molecular weight of the feedstock, change the crystalline structure of the feedstock, and/or increase the surface area and/or porosity of the feedstock. Radiation can be by, for example electron beam, ion beam, 100 nm to 28 nm ultraviolet (UV) light, gamma or X-ray radiation. Radiation treatments and systems for treatments are discussed in U.S. Pat. No. 8,142,620 and U.S. patent application Ser. No. 12/417,731, the entire disclosures of which are incorporated herein by reference.

Each form of radiation ionizes the biomass via particular interactions, as determined by the energy of the radiation. Heavy charged particles primarily ionize matter via Coulomb scattering; furthermore, these interactions produce energetic electrons that may further ionize matter. Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium. Electrons interact via Coulomb scattering and bremsstrahlung radiation produced by changes in the velocity of electrons.

When particles are utilized, they can be neutral (uncharged), positively charged or negatively charged. When charged, the charged particles can bear a single positive or negative charge, or multiple charges, e.g., one, two, three or even four or more charges. In instances in which chain scission is desired to change the molecular structure of the carbohydrate containing material, positively charged particles may be desirable, in part, due to their acidic nature. When particles are utilized, the particles can have the mass of a resting electron, or greater, e.g., 500, 1000, 1500, or 2000 or more times the mass of a resting electron. For example, the particles can have a mass of from about 1 atomic unit to about 150 atomic units, e.g., from about 1 atomic unit to about 50 atomic units, or from about 1 to about 25, e.g., 1, 2, 3, 4, 5, 10, 12 or 15 atomic units.

Gamma radiation has the advantage of a significant penetration depth into a variety of material in the sample.

In embodiments in which the irradiating is performed with electromagnetic radiation, the electromagnetic radiation can have, e.g., energy per photon (in electron volts) of greater than 10² eV, e.g., greater than 10³, 10⁴, 10⁵, 10⁶, or even greater than 10⁷ eV. In some embodiments, the electromagnetic radiation has energy per photon of between 10⁴ and 10⁷, e.g., between 10⁵ and 10⁶ eV. The electromagnetic radiation can have a frequency of, e.g., greater than 10¹⁶ Hz, greater than 10¹⁷ Hz, 10¹⁸, 10¹⁹, 10²⁰, or even greater than 10²¹ Hz. In some embodiments, the electromagnetic radiation has a frequency of between 10¹⁸ and 10²² Hz, e.g., between 10¹⁹ to 10²¹ Hz.

Electron bombardment may be performed using an electron beam device that has a nominal energy of less than 10 MeV, e.g., less than 7 MeV, less than 5 MeV, or less than 2 MeV, e.g., from about 0.5 to 1.5 MeV, from about 0.8 to 1.8 MeV, or from about 0.7 to 1 MeV. In some implementations the nominal energy is about 500 to 800 keV.

The electron beam may have a relatively high total beam power (the combined beam power of all accelerating heads, or, if multiple accelerators are used, of all accelerators and all heads), e.g., at least 25 kW, e.g., at least 30, 40, 50, 60, 65, 70, 80, 100, 125, or 150 kW. In some cases, the power is even as high as 500 kW, 750 kW, or even 1000 kW or more. In some cases the electron beam has a beam power of 1200 kW or more, e.g., 1400, 1600, 1800, or even 300 kW.

This high total beam power is usually achieved by utilizing multiple accelerating heads. For example, the electron beam device may include two, four, or more accelerating heads. The use of multiple heads, each of which has a relatively low beam power, prevents excessive temperature rise in the material, thereby preventing burning of the material, and also increases the uniformity of the dose through the thickness of the layer of material.

It is generally preferred that the bed of biomass material has a relatively uniform thickness. In some embodiments the thickness is less than about 1 inch (e.g., less than about 0.75 inches, less than about 0.5 inches, less than about 0.25 inches, less than about 0.1 inches, between about 0.1 and 1 inch, between about 0.2 and 0.3 inches).

It is desirable to treat the material as quickly as possible. In general, it is preferred that treatment be performed at a dose rate of greater than about 0.25 Mrad per second, e.g., greater than about 0.5, 0.75, 1, 1.5, 2, 5, 7, 10, 12, 15, or even greater than about 20 Mrad per second, e.g., about 0.25 to 2 Mrad per second. Higher dose rates allow a higher throughput for a target (e.g., the desired) dose. Higher dose rates generally require higher line speeds, to avoid thermal decomposition of the material. In one implementation, the accelerator is set for 3 MeV, 50 mA beam current, and the line speed is 24 feet/minute, for a sample thickness of about 20 mm (e.g., comminuted corn cob material with a bulk density of 0.5 g/cm³).

In some embodiments, electron bombardment is performed until the material receives a total dose of at least 0.1 Mrad, 0.25 Mrad, 1 Mrad, 5 Mrad, e.g., at least 10, 20, 30 or at least 40 Mrad. In some embodiments, the treatment is performed until the material receives a dose of from about 10 Mrad to about 50 Mrad, e.g., from about 20 Mrad to about 40 Mrad, or from about 25 Mrad to about 30 Mrad. In some implementations, a total dose of 25 to 35 Mrad is preferred, applied ideally over a couple of passes, e.g., at 5 Mrad/pass with each pass being applied for about one second. Cooling methods, systems and equipment can be used before, during, after and in between radiations, for example utilizing a cooling screw conveyor and/or a cooled vibratory conveyor.

Using multiple heads as discussed above, the material can be treated in multiple passes, for example, two passes at 10 to 20 Mrad/pass, e.g., 12 to 18 Mrad/pass, separated by a few seconds of cool-down, or three passes of 7 to 12 Mrad/pass, e.g., 5 to 20 Mrad/pass, 10 to 40 Mrad/pass, 9 to 11 Mrad/pass. As discussed herein, treating the material with several relatively low doses, rather than one high dose, tends to prevent overheating of the material and also increases dose uniformity through the thickness of the material. In some implementations, the material is stirred or otherwise mixed during or after each pass and then smoothed into a uniform layer again before the next pass, to further enhance treatment uniformity.

In some embodiments, electrons are accelerated to, for example, a speed of greater than 75 percent of the speed of light, e.g., greater than 85, 90, 95, or 99 percent of the speed of light.

In some embodiments, any processing described herein occurs on lignocellulosic material that remains dry as acquired or that has been dried, e.g., using heat and/or reduced pressure. For example, in some embodiments, the cellulosic and/or lignocellulosic material has less than about 25 wt. % retained water, measured at 25° C. and at fifty percent relative humidity (e.g., less than about 20 wt. %, less than about 15 wt. %, less than about 14 wt. %, less than about 13 wt. %, less than about 12 wt. %, less than about 10 wt. %, less than about 9 wt. %, less than about 8 wt. %, less than about 7 wt. %, less than about 6 wt. %, less than about 5 wt. %, less than about 4 wt. %, less than about 3 wt. %, less than about 2 wt. %, less than about 1 wt. %, or less than about 0.5 wt. %.

In some embodiments, two or more ionizing sources can be used, such as two or more electron sources. For example, samples can be treated, in any order, with a beam of electrons, followed by gamma radiation and UV light having wavelengths from about 100 nm to about 280 nm. In some embodiments, samples are treated with three ionizing radiation sources, such as a beam of electrons, gamma radiation, and energetic UV light. The biomass is conveyed through the treatment zone where it can be bombarded with electrons.

It may be advantageous to repeat the treatment to more thoroughly reduce the recalcitrance of the biomass and/or further modify the biomass. In particular the process parameters can be adjusted after a first (e.g., second, third, fourth or more) pass depending on the recalcitrance of the material. In some embodiments, a conveyor can be used which includes a circular system where the biomass is conveyed multiple times through the various processes described above. In some other embodiments multiple treatment devices (e.g., electron beam generators) are used to treat the biomass multiple (e.g., 2, 3, 4 or more) times. In yet other embodiments, a single electron beam generator may be the source of multiple beams (e.g., 2, 3, 4 or more beams) that can be used for treatment of the biomass.

The effectiveness in changing the molecular/supermolecular structure and/or reducing the recalcitrance of the carbohydrate-containing biomass depends on the electron energy used and the dose applied, while exposure time depends on the power and dose. In some embodiments, the dose rate and total dose are adjusted so as not to destroy (e.g., char or burn) the biomass material. For example, the carbohydrates should not be damaged in the processing so that they can be released from the biomass intact, e.g. as monomeric sugars.

In some embodiments, the treatment (with any electron source or a combination of sources) is performed until the material receives a dose of at least about 0.05 Mrad, e.g., at least about 0.1, 0.25, 0.5, 0.75, 1.0, 2.5, 5.0, 7.5, 10.0, 15, 20, 25, 30, 40, 50, 60, 70, 80, 90, 100, 125, 150, 175, or 200 Mrad. In some embodiments, the treatment is performed until the material receives a dose of between 0.1-100 Mrad, 1-200, 5-200, 10-200, 5-150, 50-150 Mrad, 5-100, 5-50, 5-40, 10-50, 10-75, 15-50, 20-35 Mrad.

In some embodiments, relatively low doses of radiation are utilized, e.g., to increase the molecular weight of a cellulosic or lignocellulosic material (with any radiation source or a combination of sources described herein). For example, a dose of at least about 0.05 Mrad, e.g., at least about 0.1 Mrad or at least about 0.25, 0.5, 0.75. 1.0, 1.5, 2.0, 2.5, 3.0, 3.5, 4.0, or at least about 5.0 Mrad. In some embodiments, the irradiation is performed until the material receives a dose of between 0.1 Mrad and 2.0 Mrad, e.g., between 0.5 rad and 4.0 Mrad or between 1.0 Mrad and 3.0 Mrad.

It also can be desirable to irradiate from multiple directions, simultaneously or sequentially, in order to achieve a desired degree of penetration of radiation into the material. For example, depending on the density and moisture content of the material, such as wood, and the type of radiation source used (e.g., gamma or electron beam), the maximum penetration of radiation into the material may be only about 0.75 inch. In such cases, a thicker section (up to 1.5 inch) can be irradiated by first irradiating the material from one side, and then turning the material over and irradiating from the other side. Irradiation from multiple directions can be particularly useful with electron beam radiation, which irradiates faster than gamma radiation but typically does not achieve as great a penetration depth.

### Radiation Opaque Materials

The invention can include processing a material (e.g., lignocellulosic or cellulosic feedstock) in a vault and/or bunker that is constructed using radiation opaque materials. In some implementations, the radiation opaque materials are selected to be capable of shielding the components from X-rays with high energy (short wavelength), which can penetrate many materials. One important factor in designing a radiation shielding enclosure is the attenuation length of the materials used, which will determine the required thickness for a particular material, blend of materials, or layered structure. The attenuation length is the penetration distance at which the radiation is reduced to approximately 1/e (e=Euler's number) times that of the incident radiation. Although virtually all materials are radiation opaque if thick enough, materials containing a high compositional percentage (e.g., density) of elements that have a high Z value (atomic number) have a shorter radiation attenuation length and thus if such materials are used a thinner, lighter shielding can be provided. Examples of high Z value materials that are used in radiation shielding are tantalum and lead. Another important parameter in radiation shielding is the halving distance, which is the thickness of a particular material that will reduce gamma ray intensity by 50%. As an example for X-ray radiation with an energy of 0.1 MeV the halving thickness is about 15.1 mm for concrete and about 2.7 mm for lead, while with an X-ray energy of 1 MeV the halving thickness for concrete is about 44.45 mm and for lead is about 7.9 mm. Radiation opaque materials can be materials that are thick or thin so long as they can reduce the radiation that passes through to the other side. Thus, if it is desired that a particular enclosure have a low wall thickness, e.g., for light weight or due to size constraints, the material chosen should have a sufficient Z value and/or attenuation length so that its halving length is less than or equal to the desired wall thickness of the enclosure.

In some cases, the radiation opaque material may be a layered material, for example having a layer of a higher Z value material, to provide good shielding, and a layer of a lower Z value material to provide other properties (e.g., structural integrity, impact resistance, etc.). In some cases, the layered material may be a “graded-Z” laminate, e.g., including a laminate in which the layers provide a gradient from high-Z through successively lower-Z elements. In some cases the radiation opaque materials can be interlocking blocks, for example, lead and/or concrete blocks can be supplied by NELCO Worldwide (Burlington, Mass.), and reconfigurable vaults can be utilized.

A radiation opaque material can reduce the radiation passing through a structure (e.g., a wall, door, ceiling, enclosure, a series of these or combinations of these) formed of the material by about at least about 10%, (e.g., at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%) as compared to the incident radiation. Therefore, an enclosure made of a radiation opaque material can reduce the exposure of equipment/system/components by the same amount. Radiation opaque materials can include stainless steel, metals with Z values above 25 (e.g., lead, iron), concrete, dirt, sand and combinations thereof. Radiation opaque materials can include a barrier in the direction of the incident radiation of at least about 1 mm (e.g., 5 mm, 10 mm, 5 cm, 10 cm, 100 cm, 1 m and even at least about 10 m).

### Radiation Sources

The type of radiation used for treating a feedstock (e.g., a lignocellulosic or cellulosic material) determines the kinds of radiation sources used as well as the radiation devices and associated equipment. The methods, systems and equipment described herein, for example for treating materials with radiation, can utilized sources as described herein as well as any other useful source.

Sources of gamma rays include radioactive nuclei, such as isotopes of cobalt, calcium, technetium, chromium, gallium, indium, iodine, iron, krypton, samarium, selenium, sodium, thallium, and xenon.

Sources of X-rays include electron beam collision with metal targets, such as tungsten or molybdenum or alloys, or compact light sources, such as those produced commercially by Lyncean.

Alpha particles are identical to the nucleus of a helium atom and are produced by the alpha decay of various radioactive nuclei, such as isotopes of bismuth, polonium, astatine, radon, francium, radium, several actinides, such as actinium, thorium, uranium, neptunium, curium, californium, americium, and plutonium.

Sources for ultraviolet radiation include deuterium or cadmium lamps.

Sources for infrared radiation include sapphire, zinc, or selenide window ceramic lamps.

Sources for microwaves include klystrons, Slevin type RF sources, or atom beam sources that employ hydrogen, oxygen, or nitrogen gases.

Accelerators used to accelerate the particles can be electrostatic DC, electrodynamic DC, RF linear, magnetic induction linear or continuous wave. For example, cyclotron type accelerators are available from IBA, Belgium, such as the RHODOTRON™ system, while DC type accelerators are available from RDI, now IBA Industrial, such as the DYNAMITRON®. Ions and ion accelerators are discussed in Introductory Nuclear Physics, Kenneth S. Krane, John Wiley & Sons, Inc. (1988), Krsto Prelec, FIZIKA B 6 (1997) 4, 177-206, Chu, William T., “Overview of Light-Ion Beam Therapy”, Columbus-Ohio, ICRU-IAEA Meeting, 18-20 Mar. 2006, Iwata, Y. et al., “Alternating-Phase-Focused IH-DTL for Heavy-Ion Medical Accelerators”, Proceedings of EPAC 2006, Edinburgh, Scotland, and Leitner, C. M. et al., “Status of the Superconducting ECR Ion Source Venus”, Proceedings of EPAC 2000, Vienna, Austria.

Electrons may be produced by radioactive nuclei that undergo beta decay, such as isotopes of iodine, cesium, technetium, and iridium. Alternatively, an electron gun can be used as an electron source via thermionic emission and accelerated through an accelerating potential. An electron gun generates electrons, which are then accelerated through a large potential (e.g., greater than about 500 thousand, greater than about 1 million, greater than about 2 million, greater than about 5 million, greater than about 6 million, greater than about 7 million, greater than about 8 million, greater than about 9 million, or even greater than 10 million volts) and then scanned magnetically in the x-y plane, where the electrons are initially accelerated in the z direction down the accelerator tube and extracted through a foil window. Scanning the electron beams is useful for increasing the irradiation surface when irradiating materials, e.g., a biomass, that is conveyed through the scanned beam. Scanning the electron beam also distributes the thermal load homogenously on the window and helps reduce the foil window rupture due to local heating by the electron beam. Window foil rupture is a cause of significant down-time due to subsequent necessary repairs and re-starting the electron gun.

Various other irradiating devices may be used in the methods disclosed herein, including field ionization sources, electrostatic ion separators, field ionization generators, thermionic emission sources, microwave discharge ion sources, recirculating or static accelerators, dynamic linear accelerators, van de Graaff accelerators, and folded tandem accelerators. Such devices are disclosed, for example, in U.S. Pat. No. 7,931,784 to Medoff, the complete disclosure of which is incorporated herein by reference.

A beam of electrons can be used as the radiation source. A beam of electrons has the advantages of high dose rates (e.g., 1, 5, or even 10 Mrad per second), high throughput, less containment, and less confinement equipment. Electron beams can also have high electrical efficiency (e.g., 80%), allowing for lower energy usage relative to other radiation methods, which can translate into a lower cost of operation and lower greenhouse gas emissions corresponding to the smaller amount of energy used. Electron beams can be generated, e.g., by electrostatic generators, cascade generators, transformer generators, low energy accelerators with a scanning system, low energy accelerators with a linear cathode, linear accelerators, and pulsed accelerators.

Electrons can also be more efficient at causing changes in the molecular structure of carbohydrate-containing materials, for example, by the mechanism of chain scission. In addition, electrons having energies of 0.5-10 MeV can penetrate low density materials, such as the biomass materials described herein, e.g., materials having a bulk density of less than 0.5 g/cm³, and a depth of 0.3-10 cm. Electrons as an ionizing radiation source can be useful, e.g., for relatively thin piles, layers or beds of materials, e.g., less than about 0.5 inch, e.g., less than about 0.4 inch, 0.3 inch, 0.25 inch, or less than about 0.1 inch. In some embodiments, the energy of each electron of the electron beam is from about 0.3 MeV to about 2.0 MeV (million electron volts), e.g., from about 0.5 MeV to about 1.5 MeV, or from about 0.7 MeV to about 1.25 MeV. Methods of irradiating materials are discussed in U.S. Pat. App. Pub. 2012/0100577 A1, filed Oct. 18, 2011, the entire disclosure of which is herein incorporated by reference.

Electron beam irradiation devices may be procured commercially from Ion Beam Applications, Louvain-la-Neuve, Belgium, NHV Corporation, Japan or the Titan Corporation, San Diego, Calif. Typical electron energies can be 0.5 MeV, 1 MeV, 2 MeV, 4.5 MeV, 7.5 MeV, or 10 MeV. Typical electron beam irradiation device power can be 1 kW, 5 kW, 10 kW, 20 kW, 50 kW, 60 kW, 70 kW, 80 kW, 90 kW, 100 kW, 125 kW, 150 kW, 175 kW, 200 kW, 250 kW, 300 kW, 350 kW, 400 kW, 450 kW, 500 kW, 600 kW, 700 kW, 800 kW, 900 kW or even 1000 kW.

Tradeoffs in considering electron beam irradiation device power specifications include cost to operate, capital costs, depreciation, and device footprint. Tradeoffs in considering exposure dose levels of electron beam irradiation would be energy costs and environment, safety, and health (ESH) concerns. Typically, generators are housed in a vault, e.g., of lead or concrete, especially for production from X-rays that are generated in the process. Tradeoffs in considering electron energies include energy costs.

The electron beam irradiation device can produce either a fixed beam or a scanning beam. A scanning beam may be advantageous with large scan sweep length and high scan speeds, as this would effectively replace a large, fixed beam width. Further, available sweep widths of 0.5 m, 1 m, 2 m or more are available. The scanning beam is preferred in most embodiments describe herein because of the larger scan width and reduced possibility of local heating and failure of the windows.

### Electron Guns—Windows

The extraction system for an electron accelerator that can be utilized for treating a feedstock (e.g., a lignocellulosic or cellulosic material) can include two window foils. The cooling gas in the two foil window extraction system can be a purge gas or a mixture, for example air, or a pure gas. In one embodiment the gas is an inert gas such as nitrogen, argon, helium and or carbon dioxide. It is preferred to use a gas rather than a liquid since energy losses to the electron beam are minimized. Mixtures of pure gas can also be used, either pre-mixed or mixed in line prior to impinging on the windows or in the space between the windows. The cooling gas can be cooled, for example, by using a heat exchange system (e.g., a chiller) and/or by using boil off from a condensed gas (e.g., liquid nitrogen, liquid helium). Window foils are described in PCT/US2013/64332 filed Oct. 10, 2013 the full disclosure of which is incorporated by reference herein.

### Heating and Throughput During Radiation Treatment

Several processes can occur in biomass when electrons from an electron beam interact with matter in inelastic collisions. For example, ionization of the material, chain scission of polymers in the material, cross linking of polymers in the material, oxidation of the material, generation of X-rays (“Bremsstrahlung”) and vibrational excitation of molecules (e.g., phonon generation). Without being bound to a particular mechanism, the reduction in recalcitrance can be due to several of these inelastic collision effects, for example ionization, chain scission of polymers, oxidation and phonon generation. Some of the effects (e.g., especially X-ray generation), necessitate shielding and engineering barriers, for example, enclosing the irradiation processes in a concrete (or other radiation opaque material) vault. Another effect of irradiation, vibrational excitation, is equivalent to heating up the sample. Heating the sample by irradiation can help in recalcitrance reduction, but excessive heating can destroy the material, as will be explained below.

The adiabatic temperature rise (ΔT) from adsorption of ionizing radiation is given by the equation: ΔT=D/Cp: where D is the average dose in kGy, Cp is the heat capacity in J/g ° C., and ΔT is the change in temperature in ° C. A typical dry biomass material will have a heat capacity close to 2. Wet biomass will have a higher heat capacity dependent on the amount of water since the heat capacity of water is very high (4.19 J/g ° C.). Metals have much lower heat capacities, for example 304 stainless steel has a heat capacity of 0.5 J/g ° C. The calculated temperature change due to the instant adsorption of radiation in a biomass and stainless steel for various doses of radiation is shown in Table 1. In some cases, as indicated in the table, the temperatures are so high that the material decomposes (e.g., is volatilized, carbonized, and/or charred).

TABLE 2
Calculated Temperature increase for biomass and stainless steel.

| Dose (Mrad) | Estimated Biomass ΔT (° C.) | Steel ΔT (° C.) |
|---|---|---|
| 10 | 50 | 200 |
| 50 | 250 (decomposed) | 1000 |
| 100 | 500 (decomposed) | 2000 |
| 150 | 750 (decomposed) | 3000 |
| 200 | 1000 (decomposed) | 4000 |

High temperatures can destroy and or modify the biopolymers in biomass so that the polymers (e.g., cellulose) are unsuitable for further processing. A biomass subjected to high temperatures can become dark, sticky and give off odors indicating decomposition. The stickiness can even make the material hard to convey. The odors can be unpleasant and be a safety issue. In fact, keeping the biomass below about 200° C. has been found to be beneficial in the processes described herein (e.g., below about 190° C., below about 180° C., below about 170° C., below about 160° C., below about 150° C., below about 140° C., below about 130° C., below about 120° C., below about 110° C., between about 60° C. and 180° C., between about 60° C. and 160° C., between about 60° C. and 150° C., between about 60° C. and 140° C., between about 60° C. and 130° C., between about 60° C. and 120° C., between about 80° C. and 180° C., between about 100° C. and 180° C., between about 120° C. and 180° C., between about 140° C. and 180° C., between about 160° C. and 180° C., between about 100° C. and 140° C., between about 80° C. and 120° C.).

It has been found that irradiation above about 10 Mrad is desirable for the processes described herein (e.g., reduction of recalcitrance). A high throughput is also desirable so that the irradiation does not become a bottle neck in processing the biomass. The treatment is governed by a Dose rate equation: M=FP/D·time, where M is the mass of irradiated material (kg), F is the fraction of power that is adsorbed (unit less), P is the emitted power (kW=Voltage in MeV×Current in mA), time is the treatment time (sec) and D is the adsorbed dose (kGy). In an exemplary process where the fraction of adsorbed power is fixed, the Power emitted is constant and a set dosage is desired, the throughput (e.g., M, the biomass processed) can be increased by increasing the irradiation time. However, increasing the irradiation time without allowing the material to cool, can excessively heat the material as exemplified by the calculations shown above. Since biomass has a low thermal conductivity (less than about 0.1 Wm⁻¹K⁻¹), heat dissipation is slow, unlike, for example metals (greater than about 10 Wm⁻¹K⁻¹) which can dissipate energy quickly as long as there is a heat sink to transfer the energy to.

### Electron Guns—Beam Stops

In some embodiments the systems and methods (e.g., that utilize electron beam irradiation to irradiate a lignocellulosic or cellulosic feedstock) include a beam stop (e.g., a shutter). For example, the beam stop can be used to quickly stop or reduce the irradiation of material without powering down the electron beam device. Alternatively the beam stop can be used while powering up the electron beam, e.g., the beam stop can stop the electron beam until a beam current of a desired level is achieved. The beam stop can be placed between the primary foil window and a secondary foil window. For example the beam stop can be mounted so that it is movable, that is, so that it can be moved into and out of the beam path. Even partial coverage of the beam can be used, for example, to control the dose of irradiation. The beam stop can be mounted to the floor, to a conveyor for the biomass, to a wall, to the radiation device (e.g., at the scan horn), or to any structural support. Preferably the beam stop is fixed in relation to the scan horn so that the beam can be effectively controlled by the beam stop. The beam stop can incorporate a hinge, a rail, wheels, slots, or other means allowing for its operation in moving into and out of the beam. The beam stop can be made of any material that will stop at least 5% of the electrons, e.g., at least 10%, 20%, 30%, 40%, 50%, 60%, 70%, at least 80%, 85%, 90%, 91%, 92%, 93%, 94%, 95%, 96%, 97%, 98%, 99% or even about 100% of the electrons.

The beam stop can be made of a metal including, but not limited to, stainless steel, lead, iron, molybdenum, silver, gold, titanium, aluminum, tin, or alloys of these, or laminates (layered materials) made with such metals (e.g., metal-coated ceramic, metal-coated polymer, metal-coated composite, multilayered metal materials).

The beam stop can be cooled, for example, with a cooling fluid such as an aqueous solution or a gas. The beam stop can be partially or completely hollow, for example with cavities. Interior spaces of the beam stop can be used for cooling fluids and gases. The beam stop can be of any shape, including flat, curved, round, oval, square, rectangular, beveled and wedged shapes.

The beam stop can have perforations so as to allow some electrons through, thus controlling (e.g., reducing) the levels of radiation across the whole area of the window, or in specific regions of the window. The beam stop can be a mesh formed, for example, from fibers or wires. Multiple beam stops can be used, together or independently, to control the irradiation. The beam stop can be remotely controlled, e.g., by radio signal or hard wired to a motor for moving the beam into or out of position.

### Beam Dumps

The embodiments disclosed herein (e.g., the utilize ionizing radiation to irradiate a lignocellulosic or cellulosic feedstock) can also include a beam dump when utilizing a radiation treatment. A beam dump's purpose is to safely absorb a beam of charged particles. Like a beam stop, a beam dump can be used to block the beam of charged particles. However, a beam dump is much more robust than a beam stop, and is intended to block the full power of the electron beam for an extended period of time. They are often used to block the beam as the accelerator is powering up.

Beam dumps are also designed to accommodate the heat generated by such beams, and are usually made from materials such as copper, aluminum, carbon, beryllium, tungsten, or mercury. Beam dumps can be cooled, for example, using a cooling fluid that can be in thermal contact with the beam dump.

### Biomass Materials

Lignocellulosic materials (e.g., feedstocks that are saccharified) include, but are not limited to, wood, particle board, forestry wastes (e.g., sawdust, aspen wood, wood chips), grasses, (e.g., switchgrass, miscanthus, cord grass, reed canary grass), grain residues, (e.g., rice hulls, oat hulls, wheat chaff, barley hulls), agricultural waste (e.g., silage, canola straw, wheat straw, barley straw, oat straw, rice straw, jute, hemp, flax, bamboo, sisal, abaca, corn cobs, corn stover, soybean stover, corn fiber, alfalfa, hay, coconut hair), sugar processing residues (e.g., bagasse, beet pulp, agave bagasse), algae, seaweed, manure, sewage, and mixtures of any of these.

In some cases, the lignocellulosic material includes corncobs. Ground or hammermilled corncobs can be spread in a layer of relatively uniform thickness for irradiation, and after irradiation are easy to disperse in the medium for further processing. To facilitate harvest and collection, in some cases the entire corn plant is used, including the corn stalk, corn kernels, and in some cases even the root system of the plant.

Advantageously, no additional nutrients (other than a nitrogen source, e.g., urea or ammonia) are required during fermentation of corncobs or cellulosic or lignocellulosic materials containing significant amounts of corncobs.

Corncobs, before and after comminution, are also easier to convey and disperse, and have a lesser tendency to form explosive mixtures in air than other cellulosic or lignocellulosic materials such as hay and grasses.

Cellulosic materials include, for example, paper, paper products, paper waste, paper pulp, pigmented papers, loaded papers, coated papers, filled papers, magazines, printed matter (e.g., books, catalogs, manuals, labels, calendars, greeting cards, brochures, prospectuses, newsprint), printer paper, polycoated paper, card stock, cardboard, paperboard, materials having a high a-cellulose content such as cotton, and mixtures of any of these. For example paper products as described in U.S. application Ser. No. 13/396,365 (“Magazine Feedstocks” by Medoff et al., filed Feb. 14, 2012), the full disclosure of which is incorporated herein by reference.

Cellulosic materials can also include lignocellulosic materials which have been partially or fully de-lignified.

In some instances other biomass materials can be utilized, for example starchy materials. Starchy materials include starch itself, e.g., corn starch, wheat starch, potato starch or rice starch, a derivative of starch, or a material that includes starch, such as an edible food product or a crop. For example, the starchy material can be arracacha, buckwheat, banana, barley, cassava, kudzu, oca, sago, sorghum, regular household potatoes, sweet potato, taro, yams, or one or more beans, such as favas, lentils or peas. Blends of any two or more starchy materials are also starchy materials. Mixtures of starchy, cellulosic and or lignocellulosic materials can also be used. For example, a biomass can be an entire plant, a part of a plant or different parts of a plant, e.g., a wheat plant, cotton plant, a corn plant, rice plant or a tree. The starchy materials can be treated by any of the methods described herein.

Microbial materials that can be used as feedstock can include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture and fermentation systems.

In other embodiments, the biomass materials, such as cellulosic, starchy and lignocellulosic feedstock materials, can be obtained from transgenic microorganisms and plants that have been modified with respect to a wild type variety. Such modifications may be, for example, through the iterative steps of selection and breeding to obtain desired traits in a plant. Furthermore, the plants can have had genetic material removed, modified, silenced and/or added with respect to the wild type variety. For example, genetically modified plants can be produced by recombinant DNA methods, where genetic modifications include introducing or modifying specific genes from parental varieties, or, for example, by using transgenic breeding wherein a specific gene or genes are introduced to a plant from a different species of plant and/or bacteria. Another way to create genetic variation is through mutation breeding wherein new alleles are artificially created from endogenous genes. The artificial genes can be created by a variety of ways including treating the plant or seeds with, for example, chemical mutagens (e.g., using alkylating agents, epoxides, alkaloids, peroxides, formaldehyde), irradiation (e.g., X-rays, gamma rays, neutrons, beta particles, alpha particles, protons, deuterons, UV radiation) and temperature shocking or other external stressing and subsequent selection techniques. Other methods of providing modified genes is through error prone PCR and DNA shuffling followed by insertion of the desired modified DNA into the desired plant or seed. Methods of introducing the desired genetic variation in the seed or plant include, for example, the use of a bacterial carrier, biolistics, calcium phosphate precipitation, electroporation, gene splicing, gene silencing, lipofection, microinjection and viral carriers. Additional genetically modified materials have been described in U.S. application Ser. No. 13/396,369 filed Feb. 14, 2012 the full disclosure of which is incorporated herein by reference.

Any of the methods described herein can be practiced with mixtures of any biomass materials described herein.

### Other Materials

Other materials (e.g., natural or synthetic materials), for example polymers, can be treated and/or made utilizing the methods, equipment and systems described herein. For example, such materials include polyethylene (e.g., linear low density ethylene and high density polyethylene), polystyrenes, sulfonated polystyrenes, poly (vinyl chloride), polyesters (e.g., nylons, DACRON™, KODEL™), polyalkylene esters, poly vinyl esters, polyamides (e.g., KEVLAR™), polyethylene terephthalate, cellulose acetate, acetal, poly acrylonitrile, polycarbonates (e.g., LEXAN™), acrylics [e.g., poly (methyl methacrylate), poly(methyl methacrylate), polyacrylonitrile], Poly urethanes, polypropylene, poly butadiene, polyisobutylene, polyacrylonitrile, polychloroprene (e.g. neoprene), poly(cis-1,4-isoprene) [e.g., natural rubber], poly(trans-1,4-isoprene) [e.g., gutta percha], phenol formaldehyde, melamine formaldehyde, epoxides, polyesters, poly amines, polycarboxylic acids, polylactic acids, polyvinyl alcohols, polyanhydrides, poly fluoro carbons (e.g., TEFLON™), silicones (e.g., silicone rubber), polysilanes, poly ethers (e.g., polyethylene oxide, polypropylene oxide), waxes, oils and mixtures of these. Also included are plastics, rubbers, elastomers, fibers, waxes, gels, oils, adhesives, thermoplastics, thermosets, biodegradable polymers, resins made with these polymers, other polymers, other materials and combinations thereof. The polymers can be made by any useful method including cationic polymerization, anionic polymerization, radical polymerization, metathesis polymerization, ring opening polymerization, graft polymerization, addition polymerization. In some cases the treatments disclosed herein can be used, for example, for radically initiated graft polymerization and cross linking. Composites of polymers, for example with glass, metals, biomass (e.g., fibers, particles), ceramics can also be treated and/or made.

Other materials that can be treated by using the methods, systems and equipment disclosed herein are ceramic materials, minerals, metals, inorganic compounds. For example, silicon and germanium crystals, silicon nitrides, metal oxides, semiconductors, insulators, cements and or conductors.

In addition, manufactured multipart or shaped materials (e.g., molded, extruded, welded, riveted, layered or combined in any way) can be treated, for example cables, pipes, boards, enclosures, integrated semiconductor chips, circuit boards, wires, tires, windows, laminated materials, gears, belts, machines, combinations of these. For example, treating a material by the methods described herein can modify the surfaces, for example, making them susceptible to further functionalization, combinations (e.g., welding) and/or treatment can cross link the materials.

For example, such materials can be mixed in with a lignocellulosic or cellulosic material and or be included with the biomass feedstock.

### Biomass Material Preparation—Mechanical Treatments

The biomass can be in a dry form, for example with less than about 35% moisture content (e.g., less than about 20%, less than about 15%, less than about 10% less than about 5%, less than about 4%, less than about 3%, less than about 2% or even less than about 1%). The biomass can also be delivered in a wet state, for example as a wet solid, a slurry or a suspension with at least about 10 wt % solids (e.g., at least about 20 wt. %, at least about 30 wt. %, at least about 40 wt. %, at least about 50 wt. %, at least about 60 wt. %, at least about 70 wt. %).

The processes disclosed herein can utilize low bulk density materials, for example cellulosic or lignocellulosic feedstocks that have been physically pretreated to have a bulk density of less than about 0.75 g/cm³, e.g., less than about 0.7, 0.65, 0.60, 0.50, 0.35, 0.25, 0.20, 0.15, 0.10, 0.05 or less, e.g., less than about 0.025 g/cm³. Bulk density is determined using ASTM D1895B. Briefly, the method involves filling a measuring cylinder of known volume with a sample and obtaining a weight of the sample. The bulk density is calculated by dividing the weight of the sample in grams by the known volume of the cylinder in cubic centimeters. If desired, low bulk density materials can be densified, for example, by methods described in U.S. Pat. No. 7,971,809 to Medoff, the full disclosure of which is hereby incorporated by reference.

In some cases, the pre-treatment processing includes screening of the biomass material. Screening can be through a mesh or perforated plate with a desired opening size, for example, less than about 6.35 mm (¼ inch, 0.25 inch), (e.g., less than about 3.18 mm (⅛ inch, 0.125 inch), less than about 1.59 mm ( 1/16 inch, 0.0625 inch), is less than about 0.79 mm ( 1/32 inch, 0.03125 inch), e.g., less than about 0.51 mm ( 1/50 inch, 0.02000 inch), less than about 0.40 mm ( 1/64 inch, 0.015625 inch), less than about 0.23 mm (0.009 inch), less than about 0.20 mm ( 1/128 inch, 0.0078125 inch), less than about 0.18 mm (0.007 inch), less than about 0.13 mm (0.005 inch), or even less than about 0.10 mm ( 1/256 inch, 0.00390625 inch)). In one configuration the desired biomass falls through the perforations or screen and thus biomass larger than the perforations or screen are not irradiated. These larger materials can be re-processed, for example by comminuting, or they can simply be removed from processing. In another configuration material that is larger than the perforations is irradiated and the smaller material is removed by the screening process or recycled. In this kind of a configuration, the conveyor itself (for example a part of the conveyor) can be perforated or made with a mesh. For example, in one particular embodiment the biomass material may be wet and the perforations or mesh allow water to drain away from the biomass before irradiation.

Screening of material can also be by a manual method, for example by an operator or mechanoid (e.g., a robot equipped with a color, reflectivity or other sensor) that removes unwanted material. Screening can also be by magnetic screening wherein a magnet is disposed near the conveyed material and the magnetic material is removed magnetically.

Optional pre-treatment processing can include heating the material. For example a portion of a conveyor conveying the biomass or other material can be sent through a heated zone. The heated zone can be created, for example, by IR radiation, microwaves, combustion (e.g., gas, coal, oil, biomass), resistive heating and/or inductive coils. The heat can be applied from at least one side or more than one side, can be continuous or periodic and can be for only a portion of the material or all the material. For example, a portion of the conveying trough can be heated by use of a heating jacket. Heating can be, for example, for the purpose of drying the material. In the case of drying the material, this can also be facilitated, with or without heating, by the movement of a gas (e.g., air, oxygen, nitrogen, He, CO₂, Argon) over and/or through the biomass as it is being conveyed.

Optionally, pre-treatment processing can include cooling the material. Cooling material is described in U.S. Pat. No. 7,900,857 to Medoff, the disclosure of which in incorporated herein by reference. For example, cooling can be by supplying a cooling fluid, for example water (e.g., with glycerol), or nitrogen (e.g., liquid nitrogen) to the bottom of the conveying trough. Alternatively, a cooling gas, for example, chilled nitrogen can be blown over the biomass materials or under the conveying system.

Another optional pre-treatment processing method can include adding a material to the biomass or other feedstocks. The additional material can be added by, for example, by showering, sprinkling and or pouring the material onto the biomass as it is conveyed. Materials that can be added include, for example, metals, ceramics and/or ions as described in U.S. Pat. App. Pub. 2010/0105119 A1 (filed Oct. 26, 2009) and U.S. Pat. App. Pub. 2010/0159569 A1 (filed Dec. 16, 2009), the entire disclosures of which are incorporated herein by reference. Optional materials that can be added include acids and bases. Other materials that can be added are oxidants (e.g., peroxides, chlorates), polymers, polymerizable monomers (e.g., containing unsaturated bonds), water, catalysts, enzymes and/or organisms. Materials can be added, for example, in pure form, as a solution in a solvent (e.g., water or an organic solvent) and/or as a solution. In some cases the solvent is volatile and can be made to evaporate e.g., by heating and/or blowing gas as previously described. The added material may form a uniform coating on the biomass or be a homogeneous mixture of different components (e.g., biomass and additional material). The added material can modulate the subsequent irradiation step by increasing the efficiency of the irradiation, damping the irradiation or changing the effect of the irradiation (e.g., from electron beams to X-rays or heat). The method may have no impact on the irradiation but may be useful for further downstream processing. The added material may help in conveying the material, for example, by lowering dust levels.

Biomass can be delivered to a conveyor (e.g., vibratory conveyors used in the vaults herein described) by a belt conveyor, a pneumatic conveyor, a screw conveyor, a hopper, a pipe, manually or by a combination of these. The biomass can, for example, be dropped, poured and/or placed onto the conveyor by any of these methods. In some embodiments the material is delivered to the conveyor using an enclosed material distribution system to help maintain a low oxygen atmosphere and/or control dust and fines. Lofted or air suspended biomass fines and dust are undesirable because these can form an explosion hazard or damage the window foils of an electron gun (if such a device is used for treating the material).

The material can be leveled to form a uniform thickness between about 0.0312 and 5 inches (e.g., between about 0.0625 and 2.000 inches, between about 0.125 and 1 inches, between about 0.125 and 0.5 inches, between about 0.3 and 0.9 inches, between about 0.2 and 0.5 inches between about 0.25 and 1.0 inches, between about 0.25 and 0.5 inches, 0.100+/−0.025 inches, 0.150+/−0.025 inches, 0.200+/−0.025 inches, 0.250+/−0.025 inches, 0.300+/−0.025 inches, 0.350+/−0.025 inches, 0.400+/−0.025 inches, 0.450+/−0.025 inches, 0.500+/−0.025 inches, 0.550+/−0.025 inches, 0.600+/−0.025 inches, 0.700+/−0.025 inches, 0.750+/−0.025 inches, 0.800+/−0.025 inches, 0.850+/−0.025 inches, 0.900+/−0.025 inches, 0.900+/−0.025 inches.

Generally, it is preferred to convey the material as quickly as possible through the electron beam to maximize throughput. For example the material can be conveyed at rates of at least 1 ft/min, e.g., at least 2 ft/min, at least 3 ft/min, at least 4 ft/min, at least 5 ft/min, at least 10 ft/min, at least 15 ft/min, 20, 25, 30, 35, 40, 45, 50 ft/min. The rate of conveying is related to the beam current, for example, for a ¼ inch thick biomass and 100 mA, the conveyor can move at about 20 ft/min to provide a useful irradiation dosage, at 50 mA the conveyor can move at about 10 ft/min to provide approximately the same irradiation dosage.

After the biomass material has been conveyed through the radiation zone, optional post-treatment processing can be done. The optional post-treatment processing can, for example, be a process described with respect to the pre-irradiation processing. For example, the biomass can be screened, heated, cooled, and/or combined with additives. Uniquely to post-irradiation, quenching of the radicals can occur, for example, quenching of radicals by the addition of fluids or gases (e.g., oxygen, nitrous oxide, ammonia, liquids), using pressure, heat, and/or the addition of radical scavengers. For example, the biomass can be conveyed out of the enclosed conveyor and exposed to a gas (e.g., oxygen) where it is quenched, forming carboxylated groups. In one embodiment the biomass is exposed during irradiation to the reactive gas or fluid. Quenching of biomass that has been irradiated is described in U.S. Pat. No. 8,083,906 to Medoff, the entire disclosure of which is incorporate herein by reference.

If desired, one or more mechanical treatments can be used in addition to irradiation to further reduce the recalcitrance of the carbohydrate-containing material. These processes can be applied before, during and or after irradiation.

In some cases, the mechanical treatment may include an initial preparation of the feedstock as received, e.g., size reduction of materials, such as by comminution, e.g., cutting, grinding, shearing, pulverizing or chopping. For example, in some cases, loose feedstock (e.g., recycled paper, starchy materials, or switchgrass) is prepared by shearing or shredding. Mechanical treatment may reduce the bulk density of the carbohydrate-containing material, increase the surface area of the carbohydrate-containing material and/or decrease one or more dimensions of the carbohydrate-containing material.

Alternatively, or in addition, the feedstock material can be treated with another treatment, for example chemical treatments, such as with an acid (HCl, H₂SO₄, H₃PO₄), a base (e.g., KOH and NaOH), a chemical oxidant (e.g., peroxides, chlorates, ozone), irradiation, steam explosion, pyrolysis, sonication, oxidation, chemical treatment. The treatments can be in any order and in any sequence and combinations. For example, the feedstock material can first be physically treated by one or more treatment methods, e.g., chemical treatment including and in combination with acid hydrolysis (e.g., utilizing HCl, H₂SO₄, H₃PO₄), radiation, sonication, oxidation, pyrolysis or steam explosion, and then mechanically treated. This sequence can be advantageous since materials treated by one or more of the other treatments, e.g., irradiation or pyrolysis, tend to be more brittle and, therefore, it may be easier to further change the structure of the material by mechanical treatment. As another example, a feedstock material can be conveyed through ionizing radiation using a conveyor as described herein and then mechanically treated. Chemical treatment can remove some or all of the lignin (for example chemical pulping) and can partially or completely hydrolyze the material. The methods also can be used with pre-hydrolyzed material. The methods also can be used with material that has not been pre hydrolyzed The methods can be used with mixtures of hydrolyzed and non-hydrolyzed materials, for example with about 50% or more non-hydrolyzed material, with about 60% or more non-hydrolyzed material, with about 70% or more non-hydrolyzed material, with about 80% or more non-hydrolyzed material or even with 90% or more non-hydrolyzed material.

In addition to size reduction, which can be performed initially and/or later in processing, mechanical treatment can also be advantageous for “opening up,” “stressing,” breaking or shattering the carbohydrate-containing materials, making the cellulose of the materials more susceptible to chain scission and/or disruption of crystalline structure during the physical treatment.

Methods of mechanically treating the carbohydrate-containing material include, for example, milling or grinding. Milling may be performed using, for example, a hammer mill, ball mill, colloid mill, conical or cone mill, disk mill, edge mill, Wiley mill, grist mill or other mill. Grinding may be performed using, for example, a cutting/impact type grinder. Some exemplary grinders include stone grinders, pin grinders, coffee grinders, and burr grinders. Grinding or milling may be provided, for example, by a reciprocating pin or other element, as is the case in a pin mill. Other mechanical treatment methods include mechanical ripping or tearing, other methods that apply pressure to the fibers, and air attrition milling. Suitable mechanical treatments further include any other technique that continues the disruption of the internal structure of the material that was initiated by the previous processing steps.

Mechanical feed preparation systems can be configured to produce streams with specific characteristics such as, for example, specific maximum sizes, specific length-to-width, or specific surface areas ratios. Physical preparation can increase the rate of reactions, improve the movement of material on a conveyor, improve the irradiation profile of the material, improve the radiation uniformity of the material, or reduce the processing time required by opening up the materials and making them more accessible to processes and/or reagents, such as reagents in a solution.

The bulk density of feedstocks can be controlled (e.g., increased). In some situations, it can be desirable to prepare a low bulk density material, e.g., by densifying the material (e.g., densification can make it easier and less costly to transport to another site) and then reverting the material to a lower bulk density state (e.g., after transport). The material can be densified, for example from less than about 0.2 g/cc to more than about 0.9 g/cc (e.g., less than about 0.3 to more than about 0.5 g/cc, less than about 0.3 to more than about 0.9 g/cc, less than about 0.5 to more than about 0.9 g/cc, less than about 0.3 to more than about 0.8 g/cc, less than about 0.2 to more than about 0.5 g/cc). For example, the material can be densified by the methods and equipment disclosed in U.S. Pat. No. 7,932,065 to Medoff and International Publication No. WO 2008/073186 (which was filed Oct. 26, 2007, was published in English, and which designated the United States), the full disclosures of which are incorporated herein by reference. Densified materials can be processed by any of the methods described herein, or any material processed by any of the methods described herein can be subsequently densified.

In some embodiments, the material to be processed is in the form of a fibrous material that includes fibers provided by shearing a fiber source. For example, the shearing can be performed with a rotary knife cutter.

For example, a fiber source, e.g., that is recalcitrant or that has had its recalcitrance level reduced, can be sheared, e.g., in a rotary knife cutter, to provide a first fibrous material. The first fibrous material is passed through a first screen, e.g., having an average opening size of 1.59 mm or less ( 1/16 inch, 0.0625 inch), provide a second fibrous material. If desired, the fiber source can be cut prior to the shearing, e.g., with a shredder. For example, when a paper is used as the fiber source, the paper can be first cut into strips that are, e.g., ¼- to ½-inch wide, using a shredder, e.g., a counter-rotating screw shredder, such as those manufactured by Munson (Utica, N.Y.). As an alternative to shredding, the paper can be reduced in size by cutting to a desired size using a guillotine cutter. For example, the guillotine cutter can be used to cut the paper into sheets that are, e.g., 10 inches wide by 12 inches long.

In some embodiments, the shearing of the fiber source and the passing of the resulting first fibrous material through a first screen are performed concurrently. The shearing and the passing can also be performed in a batch-type process.

For example, a rotary knife cutter can be used to concurrently shear the fiber source and screen the first fibrous material. A rotary knife cutter includes a hopper that can be loaded with a shredded fiber source prepared by shredding a fiber source.

In some implementations, the feedstock is physically treated prior to saccharification and/or fermentation. Physical treatment processes can include one or more of any of those described herein, such as mechanical treatment, chemical treatment, irradiation, sonication, oxidation, pyrolysis or steam explosion. Treatment methods can be used in combinations of two, three, four, or even all of these technologies (in any order). When more than one treatment method is used, the methods can be applied at the same time or at different times. Other processes that change a molecular structure of a biomass feedstock may also be used, alone or in combination with the processes disclosed herein.

Mechanical treatments that may be used, and the characteristics of the mechanically treated carbohydrate-containing materials, are described in further detail in U.S. Pat. App. Pub. 2012/0100577 A1, filed Oct. 18, 2011, the full disclosure of which is hereby incorporated herein by reference.

### Sonication, Pyrolysis, Oxidation, Steam Explosion

If desired, one or more sonication, pyrolysis, oxidative, or steam explosion processes can be used instead of or in addition to irradiation and/or heating to reduce or further reduce the recalcitrance of the carbohydrate-containing material. Steam heating can optionally be utilized with the addition of acid or base. For example, these processes can be applied before, during and or after irradiation. These processes are described in detail in U.S. Pat. No. 7,932,065 to Medoff, the full disclosure of which is incorporated herein by reference.

### Intermediates and Products

Using the processes described herein, the biomass material can be converted to one or more products, such as energy, fuels, foods and materials. For example, intermediates and products such as organic acids, salts of organic acids, anhydrides, esters of organic acids and fuels, e.g., fuels for internal combustion engines or feedstocks for fuel cells. Systems and processes are described herein that can use as feedstock cellulosic and/or lignocellulosic materials that are readily available, but often can be difficult to process, e.g., municipal waste streams and waste paper streams, such as streams that include newspaper, Kraft paper, corrugated paper or mixtures of these.

Specific examples of products include, but are not limited to, hydrogen, sugars (e.g., glucose, xylose, arabinose, mannose, galactose, fructose, disaccharides, oligosaccharides and polysaccharides), alcohols (e.g., monohydric alcohols or dihydric alcohols, such as ethanol, n-propanol, isobutanol, sec-butanol, tert-butanol or n-butanol), hydrated or hydrous alcohols (e.g., containing greater than 10%, 20%, 30% or even greater than 40% water), biodiesel, organic acids, hydrocarbons (e.g., methane, ethane, propane, isobutene, pentane, n-hexane, biodiesel, bio-gasoline and mixtures thereof), co-products (e.g., proteins, such as cellulolytic proteins (enzymes) or single cell proteins), and mixtures of any of these in any combination or relative concentration, and optionally in combination with any additives (e.g., fuel additives). Other examples include carboxylic acids, salts of a carboxylic acid, a mixture of carboxylic acids and salts of carboxylic acids and esters of carboxylic acids (e.g., methyl, ethyl and n-propyl esters), ketones (e.g., acetone), aldehydes (e.g., acetaldehyde), alpha and beta unsaturated acids (e.g., acrylic acid) and olefins (e.g., ethylene). Other alcohols and alcohol derivatives include propanol, propylene glycol, 1,4-butanediol, 1,3-propanediol, sugar alcohols (e.g., erythritol, glycol, glycerol, sorbitol threitol, arabitol, ribitol, mannitol, dulcitol, fucitol, iditol, isomalt, maltitol, lactitol, xylitol and other polyols), and methyl or ethyl esters of any of these alcohols. Other products include methyl acrylate, methylmethacrylate, lactic acid, citric acid, formic acid, acetic acid, propionic acid, butyric acid, succinic acid, valeric acid, caproic acid, 3-hydroxypropionic acid, palmitic acid, stearic acid, oxalic acid, malonic acid, glutaric acid, oleic acid, linoleic acid, glycolic acid, gamma-hydroxybutyric acid, and mixtures thereof, salts of any of these acids, mixtures of any of the acids and their respective salts.

Any combination of the above products with each other, and/or of the above products with other products, which other products may be made by the processes described herein or otherwise, may be packaged together and sold as products. The products may be combined, e.g., mixed, blended or co-dissolved, or may simply be packaged or sold together.

Any of the products or combinations of products described herein may be sanitized or sterilized prior to selling the products, e.g., after purification or isolation or even after packaging, to neutralize one or more potentially undesirable contaminants that could be present in the product(s). Such sanitation can be done with electron bombardment, for example, be at a dosage of less than about 20 Mrad, e.g., from about 0.1 to 15 Mrad, from about 0.5 to 7 Mrad, or from about 1 to 3 Mrad.

The processes described herein can produce various by-product streams useful for generating steam and electricity to be used in other parts of the plant (co-generation) or sold on the open market. For example, steam generated from burning by-product streams can be used in a distillation process. As another example, electricity generated from burning by-product streams can be used to power electron beam generators used in pretreatment.

The by-products used to generate steam and electricity are derived from a number of sources throughout the process. For example, anaerobic digestion of wastewater can produce a biogas high in methane and a small amount of waste biomass (sludge). As another example, post-saccharification and/or post-distillate solids (e.g., unconverted lignin, cellulose, and hemicellulose remaining from the pretreatment and primary processes) can be used, e.g., burned, as a fuel.

Other intermediates and products, including food and pharmaceutical products, are described in U.S. Pat. App. Pub. 2010/0124583 A1, published May 20, 2010, to Medoff, the full disclosure of which is hereby incorporated by reference herein.

### Lignin Derived Products

The spent biomass (e.g., spent lignocellulosic material) from lignocellulosic processing by the methods described are expected to have a high lignin content and in addition to being useful for producing energy through combustion in a Co-Generation plant, may have uses as other valuable products. For example, the lignin can be used as captured as a plastic, or it can be synthetically upgraded to other plastics. In some instances, it can also be converted to lignosulfonates, which can be utilized as binders, dispersants, emulsifiers or sequestrants.

When used as a binder, the lignin or a lignosulfonate can, e.g., be utilized in coal briquettes, in ceramics, for binding carbon black, for binding fertilizers and herbicides, as a dust suppressant, in the making of plywood and particle board, for binding animal feeds, as a binder for fiberglass, as a binder in linoleum paste and as a soil stabilizer.

When used as a dispersant, the lignin or lignosulfonates can be used, for example in, concrete mixes, clay and ceramics, dyes and pigments, leather tanning and in gypsum board.

When used as an emulsifier, the lignin or lignosulfonates can be used, e.g., in asphalt, pigments and dyes, pesticides and wax emulsions.

As a sequestrant, the lignin or lignosulfonates can be used, e.g., in micro-nutrient systems, cleaning compounds and water treatment systems, e.g., for boiler and cooling systems.

For energy production lignin generally has a higher energy content than holocellulose (cellulose and hemicellulose) since it contains more carbon than homocellulose. For example, dry lignin can have an energy content of between about 11,000 and 12,500 BTU per pound, compared to 7,000 an 8,000 BTU per pound of holocellulose. As such, lignin can be densified and converted into briquettes and pellets for burning. For example, the lignin can be converted into pellets by any method described herein. For a slower burning pellet or briquette, the lignin can be crosslinked, such as applying a radiation dose of between about 0.5 Mrad and 5 Mrad. Crosslinking can make a slower burning form factor. The form factor, such as a pellet or briquette, can be converted to a “synthetic coal” or charcoal by pyrolyzing in the absence of air, e.g., at between 400 and 950° C. Prior to pyrolyzing, it can be desirable to crosslink the lignin to maintain structural integrity.

### Saccharification

In order to convert the feedstock to a form that can be readily processed, the glucan- or xylan-containing cellulose in the feedstock can be hydrolyzed to low molecular weight carbohydrates, such as sugars, by a saccharifying agent, e.g., an enzyme or acid, a process referred to as saccharification. The low molecular weight carbohydrates can then be used, for example, in an existing manufacturing plant, such as a single cell protein plant, an enzyme manufacturing plant, or a fuel plant, e.g., an ethanol manufacturing facility.

The feedstock can be hydrolyzed using an enzyme, e.g., by combining the materials and the enzyme in a solvent, e.g., in an aqueous solution.

Alternatively, the enzymes can be supplied by organisms that break down biomass, such as the cellulose and/or the lignin portions of the biomass, contain or manufacture various cellulolytic enzymes (cellulases), ligninases or various small molecule biomass-degrading metabolites. These enzymes may be a complex of enzymes that act synergistically to degrade crystalline cellulose or the lignin portions of biomass. Examples of cellulolytic enzymes include: endoglucanases, cellobiohydrolases, and cellobiases (beta-glucosidases).

During saccharification a cellulosic substrate can be initially hydrolyzed by endoglucanases at random locations producing oligomeric intermediates. These intermediates are then substrates for exo-splitting glucanases such as cellobiohydrolase to produce cellobiose from the ends of the cellulose polymer. Cellobiose is a water-soluble 1,4-linked dimer of glucose. Finally, cellobiase cleaves cellobiose to yield glucose. The efficiency (e.g., time to hydrolyze and/or completeness of hydrolysis) of this process depends on the recalcitrance of the cellulosic material.

Therefore, the treated biomass materials can be saccharified, generally by combining the material and a cellulase enzyme in a fluid medium, e.g., an aqueous solution. In some cases, the material is boiled, steeped, or cooked in hot water prior to saccharification, as described in U.S. Pat. App. Pub. 2012/0100577 A1 by Medoff and Masterman, published on Apr. 26, 2012, the entire contents of which are incorporated herein.

The saccharification process can be partially or completely performed in a tank (e.g., a tank having a volume of at least 4000, 40,000, or 500,000 L) in a manufacturing plant, and/or can be partially or completely performed in transit, e.g., in a rail car, tanker truck, or in a supertanker or the hold of a ship. The time required for complete saccharification will depend on the process conditions and the carbohydrate-containing material and enzyme used. If saccharification is performed in a manufacturing plant under controlled conditions, the cellulose may be substantially entirely converted to sugar, e.g., glucose in about 12-96 hours. If saccharification is performed partially or completely in transit, saccharification may take longer.

It is generally preferred that the tank contents be mixed during saccharification, e.g., using jet mixing as described in International App. No. PCT/US2010/035331, filed May 18, 2010, which was published in English as WO 2010/135380 and designated the United States, the full disclosure of which is incorporated by reference herein.

The addition of surfactants can enhance the rate of saccharification. Examples of surfactants include non-ionic surfactants, such as a Tween® 20 or Tween® 80 polyethylene glycol surfactants, ionic surfactants, or amphoteric surfactants.

It is generally preferred that the concentration of the sugar solution resulting from saccharification be relatively high, e.g., greater than 40%, or greater than 50, 60, 70, 80, 90 or even greater than 95% by weight. Water may be removed, e.g., by evaporation, to increase the concentration of the sugar solution. This reduces the volume to be shipped, and also inhibits microbial growth in the solution.

Alternatively, sugar solutions of lower concentrations may be used, in which case it may be desirable to add an antimicrobial additive, e.g., a broad spectrum antibiotic, in a low concentration, e.g., 50 to 150 ppm. Other suitable antibiotics include amphotericin B, ampicillin, chloramphenicol, ciprofloxacin, gentamicin, hygromycin B, kanamycin, neomycin, penicillin, puromycin, streptomycin. Antibiotics will inhibit growth of microorganisms during transport and storage, and can be used at appropriate concentrations, e.g., between 15 and 1000 ppm by weight, e.g., between 25 and 500 ppm, or between 50 and 150 ppm. If desired, an antibiotic can be included even if the sugar concentration is relatively high. Alternatively, other additives with anti-microbial of preservative properties may be used. Preferably the antimicrobial additive(s) are food-grade.

A relatively high concentration solution can be obtained by limiting the amount of water added to the carbohydrate-containing material with the enzyme. The concentration can be controlled, e.g., by controlling how much saccharification takes place. For example, concentration can be increased by adding more carbohydrate-containing material to the solution. In order to keep the sugar that is being produced in solution, a surfactant can be added, e.g., one of those discussed above. Solubility can also be increased by increasing the temperature of the solution. For example, the solution can be maintained at a temperature of 40-50° C., 60-80° C., or even higher.

### Saccharifying Agents

Suitable cellulolytic enzymes include cellulases from species in the genera Bacillus, Coprinus, Myceliophthora, Cephalosporium, Scytalidium, Penicillium, Aspergillus, Pseudomonas, Humicola, Fusarium, Thielavia, Acremonium, Chrysosporium and Trichoderma, especially those produced by a strain selected from the species Aspergillus (see, e.g., EP Pub. No. 0 458 162), Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium sp. (including, but not limited to, A. persicinum, A. acremonium, A. brachypenium, A. dichromosporum, A. obclavatum, A. pinkertoniae, A. roseogriseum, A. incoloratum, and A. furatum). Preferred strains include Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H. Cellulolytic enzymes may also be obtained from Chrysosporium, preferably a strain of Chrysosporium lucknowense. Additional strains that can be used include, but are not limited to, Trichoderma (particularly T. viride, T. reesei, and T. koningii), alkalophilic Bacillus (see, for example, U.S. Pat. No. 3,844,890 and EP Pub. No. 0 458 162), and Streptomyces (see, e.g., EP Pub. No. 0 458 162).

In addition to or in combination to enzymes, acids, bases and other chemicals (e.g., oxidants) can be utilized to saccharify lignocellulosic and cellulosic materials. These can be used in any combination or sequence (e.g., before, after and/or during addition of an enzyme). For example strong mineral acids can be utilized (e.g. HCl, H₂SO₄, H₃PO₄) and strong bases (e.g., NaOH, KOH).

### Sugars

In the processes described herein, for example after saccharification, sugars (e.g., glucose and xylose) can be isolated. For example sugars can be isolated by precipitation, crystallization, chromatography (e.g., simulated moving bed chromatography, high pressure chromatography), centrifugation, extraction, any other isolation method known in the art, and combinations thereof.

### Hydrogenation and Other Chemical Transformations

The processes described herein can include hydrogenation. For example glucose and xylose can be hydrogenated to sorbitol and xylitol respectively. Hydrogenation can be accomplished by use of a catalyst (e.g., Pt/gamma-Al₂O₃, Ru/C, Raney Nickel, or other catalysts know in the art) in combination with H₂ under high pressure (e.g., 10 to 12000 psi, 100 to 10000 psi). Other types of chemical transformation of the products from the processes described herein can be used, for example production of organic sugar derived products such (e.g., furfural and furfural-derived products). Chemical transformations of sugar derived products are described in U.S. Ser. No. 13/934,704 filed Jul. 3, 2013, the entire disclosure of which is incorporated herein by reference in its entirety.

### Fermentation

Yeast and Zymomonas bacteria, for example, can be used for fermentation or conversion of sugar(s) to alcohol(s). Other microorganisms are discussed below. The optimum pH for fermentations is about pH 4 to 7. For example, the optimum pH for yeast is from about pH 4 to 5, while the optimum pH for Zymomonas is from about pH 5 to 6. Typical fermentation times are about 24 to 168 hours (e.g., 24 to 96 hrs) with temperatures in the range of 20° C. to 40° C. (e.g., 26° C. to 40° C.), however thermophilic microorganisms prefer higher temperatures.

In some embodiments, e.g., when anaerobic organisms are used, at least a portion of the fermentation is conducted in the absence of oxygen, e.g., under a blanket of an inert gas such as N₂, Ar, He, CO₂ or mixtures thereof. Additionally, the mixture may have a constant purge of an inert gas flowing through the tank during part of or all of the fermentation. In some cases, anaerobic conditions can be achieved or maintained by carbon dioxide production during the fermentation and no additional inert gas is needed.

In some embodiments, all or a portion of the fermentation process can be interrupted before the low molecular weight sugar is completely converted to a product (e.g., ethanol). The intermediate fermentation products include sugar and carbohydrates in high concentrations. The sugars and carbohydrates can be isolated via any means known in the art. These intermediate fermentation products can be used in preparation of food for human or animal consumption. Additionally or alternatively, the intermediate fermentation products can be ground to a fine particle size in a stainless-steel laboratory mill to produce a flour-like substance. Jet mixing may be used during fermentation, and in some cases saccharification and fermentation are performed in the same tank.

Nutrients for the microorganisms may be added during saccharification and/or fermentation, for example the food-based nutrient packages described in U.S. Pat. App. Pub. 2012/0052536, filed Jul. 15, 2011, the complete disclosure of which is incorporated herein by reference.

“Fermentation” includes the methods and products that are disclosed in applications No. PCT/US2012/71093 published Jun. 27, 2013, PCT/US2012/71907 published Jun. 27, 2012, and PCT/US2012/71083 published Jun. 27, 2012 the contents of which are incorporated by reference herein in their entirety.

Mobile fermenters can be utilized, as described in International App. No. PCT/US2007/074028 (which was filed Jul. 20, 2007, was published in English as WO 2008/011598 and designated the United States) and has a US issued U.S. Pat. No. 8,318,453, the contents of which are incorporated herein in its entirety. Similarly, the saccharification equipment can be mobile. Further, saccharification and/or fermentation may be performed in part or entirely during transit.

### Fermentation Agents

The microorganism(s) used in fermentation can be naturally-occurring microorganisms and/or engineered microorganisms. For example, the microorganism can be a bacterium (including, but not limited to, e.g., a cellulolytic bacterium), a fungus, (including, but not limited to, e.g., a yeast), a plant, a protist, e.g., a protozoa or a fungus-like protest (including, but not limited to, e.g., a slime mold), or an alga. When the organisms are compatible, mixtures of organisms can be utilized.

Suitable fermenting microorganisms have the ability to convert carbohydrates, such as glucose, fructose, xylose, arabinose, mannose, galactose, oligosaccharides or polysaccharides into fermentation products. Fermenting microorganisms include strains of the genus Saccharomyces spp. (including, but not limited to, S. cerevisiae (baker's yeast), S. distaticus, S. uvarum), the genus Kluyveromyces, (including, but not limited to, K. marxianus, K. fragilis), the genus Candida (including, but not limited to, C. pseudotropicalis, and C. brassicae), Pichia stipitis (a relative of Candida shehatae), the genus Clavispora (including, but not limited to, C. lusitaniae and C. opuntiae), the genus Pachysolen (including, but not limited to, P. tannophilus), the genus Bretannomyces (including, but not limited to, e.g., B. clausenii (Philippidis, G. P., 1996, Cellulose bioconversion technology, in Handbook on Bioethanol: Production and Utilization, Wyman, C. E., ed., Taylor & Francis, Washington, D.C., 179-212)). Other suitable microorganisms include, for example, Zymomonas mobilis, Clostridium spp. (including, but not limited to, C. thermocellum (Philippidis, 1996, supra), C. saccharobutylacetonicum, C. tyrobutyricum C. saccharobutylicum, C. Puniceum, C. beijernckii, and C. acetobutylicum), Moniliella spp. (including but not limited to M. pollinis, M. tomentosa, M. madida, M. nigrescens, M. oedocephali, M. megachiliensis), Yarrowia lipolytica, Aureobasidium sp., Trichosporonoides sp., Trigonopsis variabilis, Trichosporon sp., Moniliellaacetoabutans sp., Typhula variabilis, Candida magnoliae, Ustilaginomycetes sp., Pseudozyma tsukubaensis, yeast species of genera Zygosaccharomyces, Debaryomyces, Hansenula and Pichia, and fungi of the dematioid genus Torula (e.g., T. corallina).

Many such microbial strains are publicly available, either commercially or through depositories such as the ATCC (American Type Culture Collection, Manassas, Va., USA), the NRRL (Agricultural Research Service Culture Collection, Peoria, Ill., USA), or the DSMZ (Deutsche Sammlung von Mikroorganismen und Zellkulturen GmbH, Braunschweig, Germany), to name a few.

Commercially available yeasts include, for example, RED STAR®/Lesaffre Ethanol Red (available from Red Star/Lesaffre, USA), FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties).

### Distillation

After fermentation, the resulting fluids can be distilled using, for example, a “beer column” to separate ethanol and other alcohols from the majority of water and residual solids. The distillation can be done under vacuum (e.g., to reduce decomposition of products in the solution such as sugars) The vapor exiting the beer column can beat least 35% by weight (e.g., at least 40%, at least 50% or at least 90% by weight) ethanol and can be fed to a rectification column. A mixture of nearly azeotropic (e.g., at least about 92.5% ethanol and water from the rectification column can be purified to pure (e.g., at least about 99.5% or even about 100%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Hydrocarbon-Containing Materials

In other embodiments utilizing the methods and systems described herein, hydrocarbon-containing materials can be processed. Any process described herein can be used to treat any hydrocarbon-containing material herein described. “Hydrocarbon-containing materials,” as used herein, is meant to include oil sands, oil shale, tar sands, coal dust, coal slurry, bitumen, various types of coal, and other naturally-occurring and synthetic materials that include both hydrocarbon components and solid matter. The solid matter can include rock, sand, clay, stone, silt, drilling slurry, or other solid organic and/or inorganic matter. The term can also include waste products such as drilling waste and by-products, refining waste and by-products, or other waste products containing hydrocarbon components, such as asphalt shingling and covering, asphalt pavement, etc.

In yet other embodiments utilizing the methods and systems described herein, wood and wood containing produces can be processed. For example lumber products can be processed, e.g. boards, sheets, laminates, beams, particle boards, composites, rough cut wood, soft wood and hard wood. In addition cut trees, bushes, wood chips, saw dust, roots, bark, stumps, decomposed wood and other wood containing biomass material can be processed.

### Conveying Systems

Various conveying systems can be used to convey the biomass material, for example, to a vault and under an electron beam in a vault. Exemplary conveyors are belt conveyors, pneumatic conveyors, screw conveyors, carts, trains, trains or carts on rails, elevators, front loaders, backhoes, cranes, various scrapers and shovels, trucks, and throwing devices can be used. For example, vibratory conveyors can be used in various processes described herein. Vibratory conveyors are described in PCT/US2013/64289 filed Oct. 10, 2013 the full disclosure of which is incorporated by reference herein.

Optionally, one or more conveying systems can be enclosed. When using an enclosure, the enclosed conveyor can also be purged with an inert gas so as to maintain an atmosphere at a reduced oxygen level. Keeping oxygen levels low avoids the formation of ozone which in some instances is undesirable due to its reactive and toxic nature. For example the oxygen can be less than about 20% (e.g., less than about 10%, less than about 1%, less than about 0.1%, less than about 0.01%, or even less than about 0.001% oxygen). Purging can be done with an inert gas including, but not limited to, nitrogen, argon, helium or carbon dioxide. This can be supplied, for example, from a boil off of a liquid source (e.g., liquid nitrogen or helium), generated or separated from air in situ, or supplied from tanks. The inert gas can be recirculated and any residual oxygen can be removed using a catalyst, such as a copper catalyst bed. Alternatively, combinations of purging, recirculating and oxygen removal can be done to keep the oxygen levels low.

The enclosed conveyor can also be purged with a reactive gas that can react with the biomass. This can be done before, during or after the irradiation process. The reactive gas can be, but is not limited to, nitrous oxide, ammonia, oxygen, ozone, hydrocarbons, aromatic compounds, amides, peroxides, azides, halides, oxyhalides, phosphides, phosphines, arsines, sulfides, thiols, boranes and/or hydrides. The reactive gas can be activated in the enclosure, e.g., by irradiation (e.g., electron beam, UV irradiation, microwave irradiation, heating, IR radiation), so that it reacts with the biomass. The biomass itself can be activated, for example by irradiation. Preferably the biomass is activated by the electron beam, to produce radicals which then react with the activated or unactivated reactive gas, e.g., by radical coupling or quenching.

Purging gases supplied to an enclosed conveyor can also be cooled, for example below about 25° C., below about 0° C., below about −40° C., below about −80° C., below about −120° C. For example, the gas can be boiled off from a compressed gas such as liquid nitrogen or sublimed from solid carbon dioxide. As an alternative example, the gas can be cooled by a chiller or part of or the entire conveyor can be cooled.

### Other Embodiments

Any material, processes or processed materials discussed herein can be used to make products and/or intermediates such as composites, fillers, binders, plastic additives, adsorbents and controlled release agents. The methods can include densification, for example, by applying pressure and heat to the materials. For example composites can be made by combining fibrous materials with a resin or polymer. For example radiation cross-linkable resin, e.g., a thermoplastic resin can be combined with a fibrous material to provide a fibrous material/cross-linkable resin combination. Such materials can be, for example, useful as building materials, protective sheets, containers and other structural materials (e.g., molded and/or extruded products). Absorbents can be, for example, in the form of pellets, chips, fibers and/or sheets. Adsorbents can be used, for example, as pet bedding, packaging material or in pollution control systems. Controlled release matrices can also be the form of, for example, pellets, chips, fibers and or sheets. The controlled release matrices can, for example, be used to release drugs, biocides, fragrances. For example, composites, absorbents and control release agents and their uses are described in U.S. Serial No. PCT/US2006/010648, filed Mar. 23, 2006, and U.S. Pat. No. 8,074,910 filed Nov. 22, 2011, the entire disclosures of which are herein incorporated by reference.

In some instances the biomass material is treated at a first level to reduce recalcitrance, e.g., utilizing accelerated electrons, to selectively release one or more sugars (e.g., xylose). The biomass can then be treated to a second level to release one or more other sugars (e.g., glucose). Optionally the biomass can be dried between treatments. The treatments can include applying chemical and biochemical treatments to release the sugars. For example, a biomass material can be treated to a level of less than about 20 Mrad (e.g., less than about 15 Mrad, less than about 10 Mrad, less than about 5 Mrad, less than about 2 Mrad) and then treated with a solution of sulfuric acid, containing less than 10% sulfuric acid (e.g., less than about 9%, less than about 8%, less than about 7%, less than about 6%, less than about 5%, less than about 4%, less than about 3%, less than about 2%, less than about 1%, less than about 0.75%, less than about 0.50%, less than about 0.25%) to release xylose. Xylose, for example that is released into solution, can be separated from solids and optionally the solids washed with a solvent/solution (e.g., with water and/or acidified water). Optionally, the solids can be dried, for example in air and/or under vacuum optionally with heating (e.g., below about 150 DEG C., below about 120 DEG C.) to a water content below about 25 wt % (below about 20 wt. %, below about 15 wt. %, below about 10 wt. %, below about 5 wt. %). The solids can then be treated with a level of less than about 30 Mrad (e.g., less than about 25 Mrad, less than about 20 Mrad, less than about 15 Mrad, less than about 10 Mrad, less than about 5 Mrad, less than about 1 Mrad or even not at all) and then treated with an enzyme (e.g., a cellulase) to release glucose. The glucose (e.g., glucose in solution) can be separated from the remaining solids. The solids can then be further processed, for example utilized to make energy or other products (e.g., lignin derived products).

### Flavors, Fragrances and Colorants

Any of the products and/or intermediates described herein, for example, produced by the processes, systems and/or equipment described herein, can be combined with flavors, fragrances, colorants and/or mixtures of these. For example, any one or more of (optionally along with flavors, fragrances and/or colorants) sugars, organic acids, fuels, polyols, such as sugar alcohols, biomass, fibers and composites can be combined with (e.g., formulated, mixed or reacted) or used to make other products. For example, one or more such product can be used to make soaps, detergents, candies, drinks (e.g., cola, wine, beer, liquors such as gin or vodka, sports drinks, coffees, teas), pharmaceuticals, adhesives, sheets (e.g., woven, none woven, filters, tissues) and/or composites (e.g., boards). For example, one or more such product can be combined with herbs, flowers, petals, spices, vitamins, potpourri, or candles. For example, the formulated, mixed or reacted combinations can have flavors/fragrances of grapefruit, orange, apple, raspberry, banana, lettuce, celery, cinnamon, chocolate, vanilla, peppermint, mint, onion, garlic, pepper, saffron, ginger, milk, wine, beer, tea, lean beef, fish, clams, olive oil, coconut fat, pork fat, butter fat, beef bouillon, legume, potatoes, marmalade, ham, coffee and cheeses.

Flavors, fragrances and colorants can be added in any amount, such as between about 0.001 wt. % to about 30 wt. %, e.g., between about 0.01 to about 20, between about 0.05 to about 10, or between about 0.1 wt. % to about 5 wt. %. These can be formulated, mixed and or reacted (e.g., with any one of more product or intermediate described herein) by any means and in any order or sequence (e.g., agitated, mixed, emulsified, gelled, infused, heated, sonicated, and/or suspended). Fillers, binders, emulsifier, antioxidants can also be utilized, for example protein gels, starches and silica.

In one embodiment the flavors, fragrances and colorants can be added to the biomass immediately after the biomass is irradiated such that the reactive sites created by the irradiation may react with reactive compatible sites of the flavors, fragrances, and colorants.

The flavors, fragrances and colorants can be natural and/or synthetic materials. These materials can be one or more of a compound, a composition or mixtures of these (e.g., a formulated or natural composition of several compounds). Optionally the flavors, fragrances, antioxidants and colorants can be derived biologically, for example, from a fermentation process (e.g., fermentation of saccharified materials as described herein). Alternatively, or additionally these flavors, fragrances and colorants can be harvested from a whole organism (e.g., plant, fungus, animal, bacteria or yeast) or a part of an organism. The organism can be collected and or extracted to provide color, flavors, fragrances and/or antioxidant by any means including utilizing the methods, systems and equipment described herein, hot water extraction, supercritical fluid extraction, chemical extraction (e.g., solvent or reactive extraction including acids and bases), mechanical extraction (e.g., pressing, comminuting, filtering), utilizing an enzyme, utilizing a bacteria such as to break down a starting material, and combinations of these methods. The compounds can be derived by a chemical reaction, for example, the combination of a sugar (e.g., as produced as described herein) with an amino acid (Maillard reaction). The flavor, fragrance, antioxidant and/or colorant can be an intermediate and or product produced by the methods, equipment or systems described herein, for example and ester and a lignin derived product.

Some examples of flavor, fragrances or colorants are polyphenols. Polyphenols are pigments responsible for the red, purple and blue colorants of many fruits, vegetables, cereal grains, and flowers. Polyphenols also can have antioxidant properties and often have a bitter taste. The antioxidant properties make these important preservatives. On class of polyphenols are the flavonoids, such as Anthocyanidines, flavanonols, flavan-3-ols, s, flavanones and flavanonols. Other phenolic compounds that can be used include phenolic acids and their esters, such as chlorogenic acid and polymeric tannins.

Among the colorants inorganic compounds, minerals or organic compounds can be used, for example titanium dioxide, zinc oxide, aluminum oxide, cadmium yellow (E.g., CdS), cadmium orange (e.g., CdS with some Se), alizarin crimson (e.g., synthetic or non-synthetic rose madder), ultramarine (e.g., synthetic ultramarine, natural ultramarine, synthetic ultramarine violet), cobalt blue, cobalt yellow, cobalt green, viridian (e.g., hydrated chromium(III)oxide), chalcophylite, conichalcite, cornubite, cornwallite and liroconite. Black pigments such as carbon black and self-dispersed blacks may be used.

Some flavors and fragrances that can be utilized include ACALEA TBHQ, ACET C-6, ALLYL AMYL GLYCOLATE, ALPHA TERPINEOL, AMBRETTOLIDE, AMBRINOL 95, ANDRANE, APHERMATE, APPLELIDE, BACDANOL®, BERGAMAL, BETA IONONE EPOXIDE, BETA NAPHTHYL ISO-BUTYL ETHER, BICYCLONONALACTONE, BORNAFIX®, CANTHOXAL, CASHMERAN®, CASHMERAN® VELVET, CASSIFFIX®, CEDRAFIX, CEDRAMBER®, CEDRYL ACETATE, CELESTOLIDE, CINNAMALVA, CITRAL DIMETHYL ACETATE, CITROLATE™, CITRONELLOL 700, CITRONELLOL 950, CITRONELLOL COEUR, CITRONELLYL ACETATE, CITRONELLYL ACETATE PURE, CITRONELLYL FORMATE, CLARYCET, CLONAL, CONIFERAN, CONIFERAN PURE, CORTEX ALDEHYDE 50% PEOMOSA, CYCLABUTE, CYCLACET®, CYCLAPROP®, CYCLEMAX™, CYCLOHEXYL ETHYL ACETATE, DAMASCOL, DELTA DAMASCONE, DIHYDRO CYCLACET, DIHYDRO MYRCENOL, DIHYDRO TERPINEOL, DIHYDRO TERPINYL ACETATE, DIMETHYL CYCLORMOL, DIMETHYL OCTANOL PQ, DIMYRCETOL, DIOLA, DIPENTENE, DULCINYL® RECRYSTALLIZED, ETHYL-3-PHENYL GLYCIDATE, FLEURAMONE, FLEURANIL, FLORAL SUPER, FLORALOZONE, FLORIFFOL, FRAISTONE, FRUCTONE, GALAXOLIDE® 50, GALAXOLIDE® 50 BB, GALAXOLIDE® 50 IPM, GALAXOLIDE® UNDILUTED, GALBASCONE, GERALDEHYDE, GERANIOL 5020, GERANIOL 600 TYPE, GERANIOL 950, GERANIOL 980 (PURE), GERANIOL CFT COEUR, GERANIOL COEUR, GERANYL ACETATE COEUR, GERANYL ACETATE, PURE, GERANYL FORMATE, GRISALVA, GUAIYL ACETATE, HELIONAL™, HERBAC, HERBALIME™, HEXADECANOLIDE, HEXALON, HEXENYL SALICYLATE CIS 3-, HYACINTH BODY, HYACINTH BODY NO. 3, HYDRATROPIC ALDEHYDE.DMA, HYDROXYOL, INDOLAROME, INTRELEVEN ALDEHYDE, INTRELEVEN ALDEHYDE SPECIAL, IONONE ALPHA, IONONE BETA, ISO CYCLO CITRAL, ISO CYCLO GERANIOL, ISO E SUPER®, ISOBUTYL QUINOLINE, JASMAL, JESSEMAL®, KHARISMAL®, KHARISMAL® SUPER, KHUSINIL, KOAVONE®, KOHINOOL®, LIFFAROME™, LIMOXAL, LINDENOL™, LYRAL®, LYRAME SUPER, MANDARIN ALD 10% TRI ETH, CITR, MARITIMA, MCK CHINESE, MEIJIFF™, MELAFLEUR, MELOZONE, METHYL ANTHRANILATE, METHYL IONONE ALPHA EXTRA, METHYL IONONE GAMMA A, METHYL IONONE GAMMA COEUR, METHYL IONONE GAMMA PURE, METHYL LAVENDER KETONE, MONTAVERDI®, MUGUESIA, MUGUET ALDEHYDE 50, MUSK Z4, MYRAC ALDEHYDE, MYRCENYL ACETATE, NECTARATE™, NEROL 900, NERYL ACETATE, OCIMENE, OCTACETAL, ORANGE FLOWER ETHER, ORIVONE, ORRINIFF 25%, OXASPIRANE, OZOFLEUR, PAMPLEFLEUR®, PEOMOSA, PHENOXANOL®, PICONIA, PRECYCLEMONE B, PRENYL ACETATE, PRISMANTOL, RESEDA BODY, ROSALVA, ROSAMUSK, SANJINOL, SANTALIFF™, SYVERTAL, TERPINEOL, TERPINOLENE 20, TERPINOLENE 90 PQ, TERPINOLENE RECT., TERPINYL ACETATE, TERPINYL ACETATE JAX, TETRAHYDRO, MUGUOL®, TETRAHYDRO MYRCENOL, TETRAMERAN, TIMBERSILK™, TOBACAROL, TRIMOFIX® O TT, TRIPLAL®, TRISAMBER®, VANORIS, VERDOX™, VERDOX™ HC, VERTENEX®, VERTENEX® HC, VERTOFIX® COEUR, VERTOLIFF, VERTOLIFF ISO, VIOLIFF, VIVALDIE, ZENOLIDE, ABS INDIA 75 PCT MIGLYOL, ABS MOROCCO 50 PCT DPG, ABS MOROCCO 50 PCT TEC, ABSOLUTE FRENCH, ABSOLUTE INDIA, ABSOLUTE MD 50 PCT BB, ABSOLUTE MOROCCO, CONCENTRATE PG, TINCTURE 20 PCT, AMBERGRIS, AMBRETTE ABSOLUTE, AMBRETTE SEED OIL, ARMOISE OIL 70 PCT THUYONE, BASIL ABSOLUTE GRAND VERT, BASIL GRAND VERT ABS MD, BASIL OIL GRAND VERT, BASIL OIL VERVEINA, BASIL OIL VIETNAM, BAY OIL TERPENELESS, BEESWAX ABS N G, BEESWAX ABSOLUTE, BENZOIN RESINOID SIAM, BENZOIN RESINOID SIAM 50 PCT DPG, BENZOIN RESINOID SIAM 50 PCT PG, BENZOIN RESINOID SIAM 70.5 PCT TEC, BLACKCURRANT BUD ABS 65 PCT PG, BLACKCURRANT BUD ABS MD 37 PCT TEC, BLACKCURRANT BUD ABS MIGLYOL, BLACKCURRANT BUD ABSOLUTE BURGUNDY, BOIS DE ROSE OIL, BRAN ABSOLUTE, BRAN RESINOID, BROOM ABSOLUTE ITALY, CARDAMOM GUATEMALA CO2 EXTRACT, CARDAMOM OIL GUATEMALA, CARDAMOM OIL INDIA, CARROT HEART, CASSIE ABSOLUTE EGYPT, CASSIE ABSOLUTE MD 50 PCT IPM, CASTOREUM ABS 90 PCT TEC, CASTOREUM ABS C 50 PCT MIGLYOL, CASTOREUM ABSOLUTE, CASTOREUM RESINOID, CASTOREUM RESINOID 50 PCT DPG, CEDROL CEDRENE, CEDRUS ATLANTICA OIL REDIST, CHAMOMILE OIL ROMAN, CHAMOMILE OIL WILD, CHAMOMILE OIL WILD LOW LIMONENE, CINNAMON BARK OIL CEYLAN, CISTE ABSOLUTE, CISTE ABSOLUTE COLORLESS, CITRONELLA OIL ASIA IRON FREE, CIVET ABS 75 PCT PG, CIVET ABSOLUTE, CIVET TINCTURE 10 PCT, CLARY SAGE ABS FRENCH DECOL, CLARY SAGE ABSOLUTE FRENCH, CLARY SAGE C'LESS 50 PCT PG, CLARY SAGE OIL FRENCH, COPAIBA BALSAM, COPAIBA BALSAM OIL, CORIANDER SEED OIL, CYPRESS OIL, CYPRESS OIL ORGANIC, DAVANA OIL, GALBANOL, GALBANUM ABSOLUTE COLORLESS, GALBANUM OIL, GALBANUM RESINOID, GALBANUM RESINOID 50 PCT DPG, GALBANUM RESINOID HERCOLYN BHT, GALBANUM RESINOID TEC BHT, GENTIANE ABSOLUTE MD 20 PCT BB, GENTIANE CONCRETE, GERANIUM ABS EGYPT MD, GERANIUM ABSOLUTE EGYPT, GERANIUM OIL CHINA, GERANIUM OIL EGYPT, GINGER OIL 624, GINGER OIL RECTIFIED SOLUBLE, GUAIACWOOD HEART, HAY ABS MD 50 PCT BB, HAY ABSOLUTE, HAY ABSOLUTE MD 50 PCT TEC, HEALINGWOOD, HYSSOP OIL ORGANIC, IMMORTELLE ABS YUGO MD 50 PCT TEC, IMMORTELLE ABSOLUTE SPAIN, IMMORTELLE ABSOLUTE YUGO, JASMIN ABS INDIA MD, JASMIN ABSOLUTE EGYPT, JASMIN ABSOLUTE INDIA, ASMIN ABSOLUTE MOROCCO, JASMIN ABSOLUTE SAMBAC, JONQUILLE ABS MD 20 PCT BB, JONQUILLE ABSOLUTE France, JUNIPER BERRY OIL FLG, JUNIPER BERRY OIL RECTIFIED SOLUBLE, LABDANUM RESINOID 50 PCT TEC, LABDANUM RESINOID BB, LABDANUM RESINOID MD, LABDANUM RESINOID MD 50 PCT BB, LAVANDIN ABSOLUTE H, LAVANDIN ABSOLUTE MD, LAVANDIN OIL ABRIAL ORGANIC, LAVANDIN OIL GROSSO ORGANIC, LAVANDIN OIL SUPER, LAVENDER ABSOLUTE H, LAVENDER ABSOLUTE MD, LAVENDER OIL COUMARIN FREE, LAVENDER OIL COUMARIN FREE ORGANIC, LAVENDER OIL MAILLETTE ORGANIC, LAVENDER OIL MT, MACE ABSOLUTE BB, MAGNOLIA FLOWER OIL LOW METHYL EUGENOL, MAGNOLIA FLOWER OIL, MAGNOLIA FLOWER OIL MD, MAGNOLIA LEAF OIL, MANDARIN OIL MD, MANDARIN OIL MD BHT, MATE ABSOLUTE BB, MOSS TREE ABSOLUTE MD TEX IFRA 43, MOSS-OAK ABS MD TEC IFRA 43, MOSS-OAK ABSOLUTE IFRA 43, MOSS-TREE ABSOLUTE MD IPM IFRA 43, MYRRH RESINOID BB, MYRRH RESINOID MD, MYRRH RESINOID TEC, MYRTLE OIL IRON FREE, MYRTLE OIL TUNISIA RECTIFIED, NARCISSE ABS MD 20 PCT BB, NARCISSE ABSOLUTE FRENCH, NEROLI OIL TUNISIA, NUTMEG OIL TERPENELESS, OEILLET ABSOLUTE, OLIBANUM RESINOID, OLIBANUM RESINOID BB, OLIBANUM RESINOID DPG, OLIBANUM RESINOID EXTRA 50 PCT DPG, OLIBANUM RESINOID MD, OLIBANUM RESINOID MD 50 PCT DPG, OLIBANUM RESINOID TEC, OPOPONAX RESINOID TEC, ORANGE BIGARADE OIL MD BHT, ORANGE BIGARADE OIL MD SCFC, ORANGE FLOWER ABSOLUTE TUNISIA, ORANGE FLOWER WATER ABSOLUTE TUNISIA, ORANGE LEAF ABSOLUTE, ORANGE LEAF WATER ABSOLUTE TUNISIA, ORRIS ABSOLUTE ITALY, ORRIS CONCRETE 15 PCT IRONE, ORRIS CONCRETE 8 PCT IRONE, ORRIS NATURAL 15 PCT IRONE 4095C, ORRIS NATURAL 8 PCT IRONE 2942C, ORRIS RESINOID, OSMANTHUS ABSOLUTE, OSMANTHUS ABSOLUTE MD 50 PCT BB, PATCHOULI HEART #3, PATCHOULI OIL INDONESIA, PATCHOULI OIL INDONESIA IRON FREE, PATCHOULI OIL INDONESIA MD, PATCHOULI OIL REDIST, PENNYROYAL HEART, PEPPERMINT ABSOLUTE MD, PETITGRAIN BIGARADE OIL TUNISIA, PETITGRAIN CITRONNIER OIL, PETITGRAIN OIL PARAGUAY TERPENELESS, PETITGRAIN OIL TERPENELESS STAB, PIMENTO BERRY OIL, PIMENTO LEAF OIL, RHODINOL EX GERANIUM CHINA, ROSE ABS BULGARIAN LOW METHYL EUGENOL, ROSE ABS MOROCCO LOW METHYL EUGENOL, ROSE ABS TURKISH LOW METHYL EUGENOL, ROSE ABSOLUTE, ROSE ABSOLUTE BULGARIAN, ROSE ABSOLUTE DAMASCENA, ROSE ABSOLUTE MD, ROSE ABSOLUTE MOROCCO, ROSE ABSOLUTE TURKISH, ROSE OIL BULGARIAN, ROSE OIL DAMASCENA LOW METHYL EUGENOL, ROSE OIL TURKISH, ROSEMARY OIL CAMPHOR ORGANIC, ROSEMARY OIL TUNISIA, SANDALWOOD OIL INDIA, SANDALWOOD OIL INDIA RECTIFIED, SANTALOL, SCHINUS MOLLE OIL, ST JOHN BREAD TINCTURE 10 PCT, STYRAX RESINOID, STYRAX RESINOID, TAGETE OIL, TEA TREE HEART, TONKA BEAN ABS 50 PCT SOLVENTS, TONKA BEAN ABSOLUTE, TUBEROSE ABSOLUTE INDIA, VETIVER HEART EXTRA, VETIVER OIL HAITI, VETIVER OIL HAITI MD, VETIVER OIL JAVA, VETIVER OIL JAVA MD, VIOLET LEAF ABSOLUTE EGYPT, VIOLET LEAF ABSOLUTE EGYPT DECOL, VIOLET LEAF ABSOLUTE FRENCH, VIOLET LEAF ABSOLUTE MD 50 PCT BB, WORMWOOD OIL TERPENELESS, YLANG EXTRA OIL, YLANG III OIL and combinations of these.

The colorants can be among those listed in the Color Index International by the Society of Dyers and Colourists. Colorants include dyes and pigments and include those commonly used for coloring textiles, paints, inks and inkjet inks. Some colorants that can be utilized include carotenoids, arylide yellows, diarylide yellows, β-naphthols, naphthols, benzimidazolones, disazo condensation pigments, pyrazolones, nickel azo yellow, phthalocyanines, quinacridones, perylenes and perinones, isoindolinone and isoindoline pigments, triarylcarbonium pigments, diketopyrrolo-pyrrole pigments, thioindigoids. Cartenoids include, for example, alpha-carotene, beta-carotene, gamma-carotene, lycopene, lutein and astaxanthin, Annatto extract, Dehydrated beets (beet powder), Canthaxanthin, Caramel, β-Apo-8′-carotenal, Cochineal extract, Carmine, Sodium copper chlorophyllin, Toasted partially defatted cooked cottonseed flour, Ferrous gluconate, Ferrous lactate, Grape color extract, Grape skin extract (enocianina), Carrot oil, Paprika, Paprika oleoresin, Mica-based pearlescent pigments, Riboflavin, Saffron, Titanium dioxide, Tomato lycopene extract; tomato lycopene concentrate, Turmeric, Turmeric oleoresin, FD&C Blue No. 1, FD&C Blue No. 2, FD&C Green No. 3, Orange B, Citrus Red No. 2, FD&C Red No. 3, FD&C Red No. 40, FD&C Yellow No. 5, FD&C Yellow No. 6, Alumina (dried aluminum hydroxide), Calcium carbonate, Potassium sodium copper chlorophyllin (chlorophyllin-copper complex), Dihydroxyacetone, Bismuth oxychloride, Ferric ammonium ferrocyanide, Ferric ferrocyanide, Chromium hydroxide green, Chromium oxide greens, Guanine, Pyrophyllite, Talc, Aluminum powder, Bronze powder, Copper powder, Zinc oxide, D&C Blue No. 4, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, D&C Violet No. 2, D&C Yellow No. 7, Ext. D&C Yellow No. 7, D&C Yellow No. 8, D&C Yellow No. 10, D&C Yellow No. 11, D&C Black No. 2, D&C Black No. 3 (3), D&C Brown No. 1, Ext. D&C, Chromium-cobalt-aluminum oxide, Ferric ammonium citrate, Pyrogallol, Logwood extract, 1,4-Bis[(2-hydroxy-ethyl)amino]-9,10-anthracenedione bis(2-propenoic)ester copolymers, 1,4-Bis [(2-methylphenyl)amino]-9,10-anthracenedione, 1,4-Bis[4-(2-methacryloxyethyl) phenylamino] anthraquinone copolymers, Carbazole violet, Chlorophyllin-copper complex, Chromium-cobalt-aluminum oxide, C.I. Vat Orange 1, 2-[[2,5-Diethoxy-4-[(4-methylphenyl)thiol] phenyl]azo]-1,3,5-benzenetriol, 16,23-Dihydrodinaphtho [2,3-a:2′,3′-i] naphth [2′,3′:6,7] indolo [2,3-c] carbazole-5,10,15,17,22,24-hexone, N,N′-(9,10-Dihydro-9,10-dioxo-1,5-anthracenediyl) bisbenzamide, 7,16-Dichloro-6,15-dihydro-5,9,14,18-anthrazinetetrone, 16,17-Dimethoxydinaphtho (1,2,3-cd:3′,2′,1′-lm) perylene-5,10-dione, Poly(hydroxyethyl methacrylate)-dye copolymers(3), Reactive Black 5, Reactive Blue 21, Reactive Orange 78, Reactive Yellow 15, Reactive Blue No. 19, Reactive Blue No. 4, C.I. Reactive Red 11, C.I. Reactive Yellow 86, C.I. Reactive Blue 163, C.I. Reactive Red 180, 4-[(2,4-dimethylphenyl)azo]-2,4-dihydro-5-methyl-2-phenyl-3H-pyrazol-3-one (solvent Yellow 18), 6-Ethoxy-2-(6-ethoxy-3-oxobenzo[b] thien-2(3H)-ylidene) benzo[b]thiophen-3(2H)-one, Phthalocyanine green, Vinyl alcohol/methyl methacrylate-dye reaction products, C.I. Reactive Red 180, C.I. Reactive Black 5, C.I. Reactive Orange 78, C.I. Reactive Yellow 15, C.I. Reactive Blue 21, Disodium 1-amino-4-[[4-[(2-bromo-1-oxoallyl)amino]-2-sulphonatophenyl]amino]-9,10-dihydro-9,10-dioxoanthracene-2-sulphonate (Reactive Blue 69), D&C Blue No. 9, [Phthalocyaninato(2-)] copper and mixtures of these.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (e.g., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of processing lignocellulosic biomass, the method comprising:
injecting a slurry comprising at least 20 percent by weight lignocellulosic biomass solids in water at a first temperature into a jet of steam at a second temperature greater than the first temperature to elevate the temperature of the biomass slurry to a desired temperature third temperature; and
saccharifying the lignocellulosic biomass slurry to produce sugars comprising xylose and glucose.

2. The method of claim 1 further comprising irradiating the lignocellulosic biomass solids prior to injecting the slurry.

3. The method of claim 2, wherein the lignocellulosic biomass solids are irradiated with between 1 and 100 Mrad of ionizing radiation.

4. The method of claim 3, wherein the lignocellulosic biomass solids are irradiated with between 20 and 40 Mrad of ionizing radiation.

5. The method of claim 1, wherein the slurry comprises at least 50 percent by weight lignocellulosic biomass solids.

6. The method of claim 1, wherein the slurry is heated for a time sufficient to swell the lignocellulosic biomass solids to at least 5 vol. % higher than un-heated lignocellulosic biomass solids.

7. The method of claim 1, wherein the slurry is heated for a time sufficient to reduce the crystallinity of the lignocellulosic biomass solids by at least 10%.

8. The method of claim 1, wherein the first temperature is below 50° C.

9. The method of claim 1, wherein the lignocellulosic biomass solids are wood, particle board, forestry wastes, grasses, grain residues, agricultural waste, sugar processing residues, algae, seaweed, manure, sewage, or mixtures of any of these.

10. The method of claim 10, wherein the lignocellulosic biomass solids are corn cobs.

11. The method of claim 1, wherein injecting the slurry into the jet of steam occurs in a Jet Cooker.

12. The method of claim 1, wherein the second temperature is between 120° C. and 350° C.

13. The method of claim 12, wherein the second temperature is between 140° C. and 250° C.

14. The method of claim 1, wherein the third temperature is between 120° C. and 160° C.

15. The method of claim 14, wherein the third temperature is between 140° C. and 180° C.
